(19) United States
(12) Patent Application Publication
(10) Pub. No.: US 2015/0024031 A1
(43) Pub. Date: Jan. 22, 2015

(54) Methods And Compositions For Reducing Pain, Inflammation, And/Or Immunological Reactions Associated With Parenterally Administering A Primary Therapeutic Agent

(71) Applicants: BAXTER INTERNATIONAL INC., Deerfield, IL (US); BAXTER HEALTHCARE SA, Glattpark (Opifkon) (CH)

(72) Inventors: Barrett Rabinow, Skokie, IL (US); Jane O. Werling, Arlington Heights, IL (US)

(73) Assignees: BAXTER INTERNATIONAL INC., Deerfield, IL (US); BAXTER HEALTHCARE SA, Glattpark (Opifkon) (CH)

(21) Appl. No.: 14/333,004

(22) Filed: Jul. 16, 2014

Related U.S. Application Data
(60) Provisional application No. 61/847,519, filed on Jul. 17, 2013.

(51) Int. Cl. A61K 31/445; A61K 31/426; A61K 9/16; A61K 31/765; A61K 38/16; A61K 9/10; A61K 31/4418; A61K 31/536

(52) U.S. Cl. CPC A61K 31/445; A61K 31/4418; A61K 31/426; A61K 31/536; A61K 31/765; A61K 38/16; A61K 9/10; A61K 9/16
USPC 424/450; 424/489; 546/335; 514/365; 514/230.5; 424/78.37; 514/114; 514/1.1; 424/484

(57) ABSTRACT

Disclosed herein are methods and pharmaceutical compositions for reducing the pain associated with parenterally administering a therapeutic agent. The methods and compositions comprise a dispersion comprising microparticles of an analgesic agent in an amount effective to reduce the pain, inflammation, and/or immunological reaction associated with parenterally administering a primary therapeutic agent, wherein the microparticles of the analgesic agent have an effective particle size of less than 20 micrometers.

### FIELD OF THE INVENTION

This invention is directed to methods and pharmaceutical compositions comprising microparticles of an analgesic agent for reducing pain, inflammation, and/or immunological reactions associated with parenteral administration of a primary therapeutic agent.

### BACKGROUND OF THE INVENTION

Parenteral dosing of therapeutic agents such as drugs, peptides, proteins, vaccines, and the like bypasses the gastrointestinal system and is therefore frequently preferred to optimize the absorption, distribution, metabolism, and/or excretion parameters of the agent. However, it is well known that parenteral administration of a therapeutic agent frequently causes pain, inflammation, and/or immunological reactions following exposure of the therapeutic agent to the cells and tissues of the body.

For example, parenteral administration of a therapeutic agent can cause sustained pain at the site of administration. For some liquid drug formulations, the pain can be attributed to the precipitation of the drug at the administration site (Alvarez-Nunez and Yalkowsky, Int J Pharm, 1999; 185(1): 45-9). Thus, parenteral administration of relatively concentrated liquid formulations and particulate drug formulations in particular can often be uncomfortable for recipients.

Parenteral dosing of a therapeutic agent can also cause local inflammation at the site of administration. When tissues are damaged, for example, by the parenteral administration of a therapeutic agent, cytokines that cause inflammation are released and can lead to an inflammatory cascade. Inflammatory cascades are generally considered to include two phases. The first phase is a cellular response, wherein white blood cells such as granulocytes, macrophages and lymphocytes are recruited to the site of injury, e.g., to clear damaged tissues, attacking bacteria, and remove noxious particles. Afterwards, there is a healing phase associated with a rebuilding of tissue and reduction in concentration of white blood cells. The increased flow of fluids, proteins, and cells to the site of injury during the first phase of the inflammatory cascade results in the symptoms typically associated with inflammation, including, but not limited to, pain, heat, swelling, erythema, and leukocyte migration. Left unchecked (as confirmed, for example, by sustained elevated leukocyte concentrations), inflammation can lead to more serious effects, such as tissue necrosis, endothelial loss, thrombosis, edema, hemorrhage, and loss of function.

Parenteral administration of a therapeutic agent can also cause a systemic immunological reaction. The systemic immunological reaction can be an adverse response to a foreign antigen, such as a hypersensitivity, typically, an allergic reaction. For example, anakinra (Kineret®, Swedish Orphan Biovitrum) is a drug used to treat rheumatoid arthritis that can cause pain at the site of administration when administered parenterally (typically, by injection) and can also trigger an allergic reaction, particularly in subjects sensitive to bacterial proteins. An adverse antigenic response may include the induction of a proliferative cellular response, the production of soluble mediators (including, but not limited to, cytokines, oxygen radicals, enzymes, prostanoids, and vasoactive amines), or cell surface expression of new or increased numbers of mediators (including, but not limited to, major histocompatability antigens and cell adhesion molecules). An adverse antigenic response can involve inflammatory cells including monocytes, macrophages, T lymphocytes, B lymphocytes, granulocytes (polymorphonuclear leukocytes including neutrophils, basophils, and eosinophils), mast cells, dendritic cells, Langerhans cells, and endothelial cells. Adverse antigenic responses can cause damage to cells and tissues, with severe and even fatal consequences.

Many approaches to reduce the potential adverse effects of parenteral administration have been tested. Such efforts have been largely focused on reducing the concentration of the drug that is in direct contact with body tissues. To date, efforts have largely focused on reducing the drug concentration that is in direct contact with body tissues by changing the formulation of a pharmaceutical composition. For example, with respect to lipid-soluble drugs formulated as emulsions, the ratio of excipient oil to drug has been increased so as to ‘sequester’ the drug away from the body tissues and encapsulate it within the interior bulk of the emulsion particle, thereby reducing the concentration of the drug on the surface of the emulsion particle and slowing the rate of uptake by body tissues in transient contact with the particle and diminishing any associated reaction (e.g., pain or inflammation) at the site of administration. Other efforts to reduce the potential adverse effects of parenteral administration involve optimizing the pH or the osmotic strength of the formulation.

The solubility of some drugs in preferred excipients for preparing emulsions such as soybean oil and lecithin is so poor, however, that relatively toxic excipients such as polyethoxylated caster oil (available from BASF under the Cremophor® and Kolliphor® trade names) are required. For example, polyethoxylated caster oil has been used, for example, to solubilize paclitaxel to facilitate parenteral administration. Generally, such formulations are undesirable because the polyethoxylated caster oil excipient itself frequently induces an allergic response. Reported symptoms include tightness in the chest, shortness of breath, and similar reactions consistent with severe anaphylactic responses.

In addition, it is known that the pH and osmolality of pharmaceutical formulations can be controlled to minimize injection site pain. For example, the pH of most pharmaceutical formulations is held between about 4.2 to 10 in order to minimize injection site pain. Similarly, the osmolality of most pharmaceutical formulations is generally held between 150 mosmol/L and 800 mosmol/L to minimize injection site pain.

Additional alternative dosage forms, including particle formulations have been proposed. Although antiretroviral drugs such as ritonavir, atazanavir, and efavirenz are typically administered orally, nanosuspension formulations thereof have been shown to be long-acting and can elicit potent antiretroviral and neuroprotective responses in subjects (Dash et al., AIDS, 2012; 26: 2135-44). However, these nanosuspension formulations frequently cause injection site reactions when administered parenterally, e.g., by subcutaneous or intramuscular injection. Because the performance or optimal efficacy of a drug formulated as a nanosuspension depends to a large degree on the characteristics of the nanosuspension particle, including its size, shape, zeta potential, and surface ligands, and each of the foregoing can exacerbate injection site reactions, the formulation criteria necessary for minimizing any adverse affects of parenteral administration must be carefully balanced against the requirements for optimal drug efficacy.

Other approaches to addressing pain and inflammation at the site of administration have relied on the co-administration of a second agent. For example, hyaluronidase has been co-administered with primary therapeutic agents such as bisphosphonates in order to degrade the connective tissue hyaluronic acid and improve the absorption of the primary therapeutic from the site of administration. Unfortunately, an unacceptable number of reactions at the site of injection were observed in subject receiving bisphosphonates.

Local anesthetic agents such as bupivacaine (typically administered by injection or infusion), ropivacaine (typically administered by injection or infusion), and lidocaine (typically administered by injection or topical application) have been used to relieve pain. However, the action of such agents is limited and subject to the drug's in vivo distribution, metabolism, and excretion. Ropivacaine hydrochloride ((S)—N-(2,6-dimethylphenyl)-1-propylpiperidine-2-carboxamide) is a local anesthetic drug belonging to the amino amide group which works by blocking nerve impulses and preventing central (spinal) pain circuits from developing. The name ropivacaine refers to both the racemate and the marketed S-enantiomer Naropin® (AstraZeneca), which is currently marketed for delivery as an injectable. Ropivacaine formulations and administration are described in EP 151110 B1, EP 239710 B1, and U.S. Pat. No. 6,620,423. Naropin® is currently indicated for surgical anesthesia via the epidural and intrathecal (spinal) routes of administration, major nerve blocks and field block infiltration. The drug is also indicated for acute pain management via the epidural route and also field blocks, intraarticular injection and continuous peripheral nerve block. A previous study (Beaussier et al., Anesthesiology, 2007; 107: 461-8) showed that continuous preperitoneal administration of 0.2% ropivacaine at 10 mL per hour for 48 hours after open colorectal resection reduced morphine consumption, improved pain relief, and accelerated postoperative recovery. As a result of that study, ropivacaine infiltration for pain control following various types of surgery has been popularized (e.g., Forastiere et al., Brit. J. Anesthesia, 2008; 101: 841-7). Post-operatively, ropivacaine is administered by continuous infusion or via a pump through a catheter running the length of incision for local pain control. The need for continuous administration of the analgesic agent in order to provide long-term relief, however, can itself be uncomfortable and inconvenient for the recipient.

The pain, inflammation, and/or immunological reactions associated with parenteral administration of a therapeutic agent can limit the clinical utility of the therapeutic agent, particularly when the primary therapeutic agent is administered in particulate form or is known to induce an allergic reaction in an unacceptable proportion of recipients. Conventional approaches to modifying pharmaceutical formulations of therapeutic agents have had limited success in combating the pain, irritation, and/or immunological reactions associated with parenteral administration. Additionally, administering analgesic agents such as local anesthetics to ameliorate the adverse effects of parenterally administering a primary therapeutic agent can be uncomfortable and inconvenient, particularly when continuous administration is indicated.

In view of the foregoing, there exists a need for compositions and methods capable of reducing the pain, inflammation, and immunological reactions associated with parenterally administering a therapeutic agent to a subject in need thereof that preferably does not cause the subject to experience pain, induce inflammation, or compromise the efficacy of the therapeutic agent.

### SUMMARY OF THE INVENTION

The invention provides methods of reducing the pain, inflammation, and/or immunological reaction associated with parenterally administering a primary therapeutic agent.

In one embodiment, a method according to the invention comprises parenterally administering to a subject in need thereof a therapeutically effective amount of a dispersion comprising microparticles of a primary therapeutic agent, the dispersion further comprising microparticles of an analgesic agent in an amount effective to reduce the pain, inflammation, and/or immunological reaction associated with parenterally administering the primary therapeutic agent, wherein the microparticles of the primary therapeutic agent and the microparticles of the analgesic agent have an effective particle size of less than 20 micrometers.

In another embodiment, a method according to the invention comprises parenterally co-administering to a subject in need thereof a therapeutically effective amount of a first dispersion comprising microparticles of a primary therapeutic agent and a second dispersion comprising microparticles of an analgesic agent, wherein the second dispersion is administered in an amount effective to reduce the pain, inflammation, and/or immunological reaction associated with parenterally administering the primary therapeutic agent, wherein the microparticles of the primary therapeutic agent and the microparticles of the analgesic agent have an effective particle size of less than 20 micrometers.

In another embodiment, a method according to the invention comprises parenterally co-administering to a subject in need thereof a therapeutically effective amount of a primary therapeutic agent and a dispersion comprising microparticles of an analgesic agent, wherein the dispersion comprising microparticles of an analgesic agent is administered in an amount effective to reduce the pain, inflammation, and/or immunological reaction associated with parenterally administering the primary therapeutic agent, wherein the microparticles of the analgesic agent have an effective particle size of less than 20 micrometers.

The invention also provides a pharmaceutical composition comprising a dispersion comprising microparticles of an analgesic agent in an amount effective to reduce the pain, inflammation, and/or immunological reaction associated with parenterally administering a primary therapeutic agent, wherein the microparticles of the analgesic agent have an effective particle size of less than 20 micrometers.

In another embodiment, a pharmaceutical composition according to the invention comprises a fibrin matrix and microparticles of an analgesic agent, said microparticles being dispersed within the fibrin matrix, wherein the microparticles of the analgesic agent have an effective particle size of less than 20 micrometers.

In an additional embodiment, the invention provides a method of preventing or reducing pain, inflammation, and/or immunological reactions in a subject suffering from arthritis, the method comprising delivering a composition according to the invention proximate to a site of arthritis, said composition being capable of releasing the analgesic agent in an amount effective for preventing or reducing pain, inflammation, and/or immunological reactions at the site of arthritis.

In another embodiment, the invention provides a method of preventing or reducing pain, inflammation, and/or immunological reactions at a site of surgery or at a wound site in a subject in need thereof, the method comprising delivering a composition according to the invention proximate to the site of surgery or the wound site, said composition being capable of releasing the analgesic agent in an amount effective for preventing or reducing pain, inflammation, and/or immunological reactions at the site of surgery or the wound site.

An analgesic agent according to the invention may comprise a drug selected from the group consisting of antihistamines, mast cell stabilizers, corticosteroids, anti-inflammatories, local anesthetics, and combinations thereof. Examples of preferred analgesic agents include local anesthetic agents such as lidocaine, mepivacaine, prilocalne, proparacaine, etidocaine, bupivacaine, levobupivacaine, ropivacaine, dibucaine, articaine, cocaine, procaine, tetracaine, articaine, benzocaine, chloroprocaine, etidocaine, pramoxine, dyclorine, benoxinate, butacaine, cyclomethycaine, hexylcaine, piperocaine, procaine, tetracaine, dibucaine, butamben, capsaicin, and combinations thereof.

### BRIEF DESCRIPTION OF THE DRAWINGS

FIG. 1 shows stability data over 12 weeks for ropivacaine formulations 1, 3, and 4. FIG. 1A shows the mean particle size of the ropivacaine suspensions. FIG. 1B shows the mean particle size of the ropivacaine suspensions and the 99% particle size of suspension 1.

FIG. 2 shows the dissolution profile of ropivacaine as measured by turbidity (NTU) over time (minutes). FIG. 2A shows the rate of ropivacaine dissolution for two concentrations of ropivacaine (˜0.25 mg/mL and ˜0.16 mg/mL) in PBS. FIG. 2B shows the rate of ropivacaine dissolution for two concentrations of ropivacaine (˜0.25 mg/mL and ˜0.38 mg/mL) in plasma.

FIG. 3 shows electron micrographs of ropivacaine microparticles in a fibrin matrix.

FIG. 4 shows the release of ropivacaine from fibrin matrices into human plasma over a period of 8 days in vitro.

FIG. 5 shows the expected ropivacaine daily release from fibrin matrices into human plasma over a period of 8 days in vivo.

FIG. 6 shows the difference in hind limb weight bearing for injected (right) and control (left) legs in rats treated with celecoxib or ropivacaine in a model of inflammation.

FIG. 7 shows the difference in foot dragging measured using gait analysis, with a higher gait analysis score indicating an increased tendency to drag the injected leg.

FIG. 8 shows the difference in hind limb weight bearing (left) and foot-dragging (right) for normal, saline (control), celecoxib, and ropivacaine treated animals.

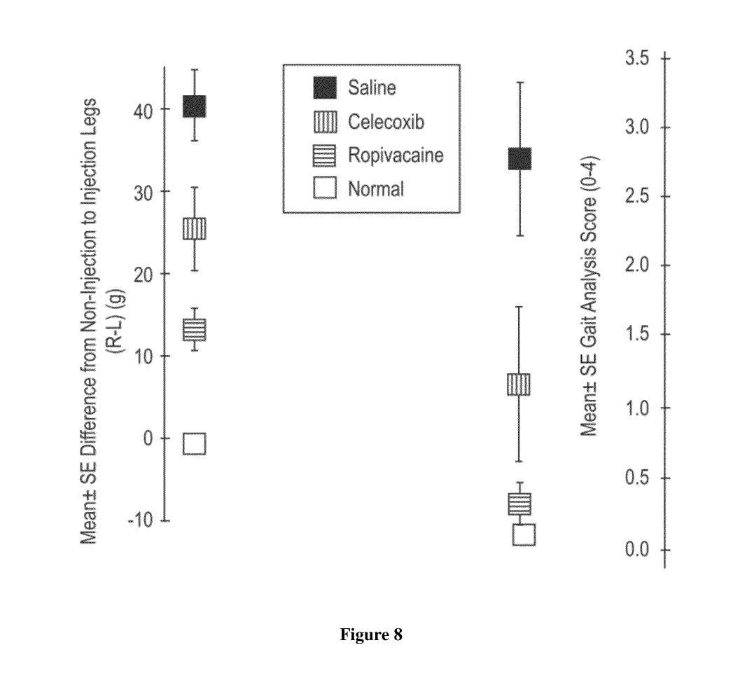

### DETAILED DESCRIPTION OF THE INVENTION

The administration of microparticles of an analgesic agent, particularly a local anesthetic agent such as ropivacaine, can beneficially reduce pain, inflammation, and/or immunological reactions, such as those associated with parenteral administration of a therapeutic agent, as demonstrated by the reduction of pain and inflammation achieved in the application Examples. Such a reduction in pain and/or inflammation is particularly useful in the context of parenterally administered sustained release formulations of a primary therapeutic agent and chronically parenterally administered drugs, which are known to cause frequent and persistent pain and irritation in subjects receiving therapy.

While addressing the pain associated with the parenteral administration of a primary therapeutic agent is an important consideration, resolving pain alone does not always indicate that inflammation is also mitigated. For some drugs, there is only a weak correlation between injection site pain and tissue damage (W. Klement, “Pain, irritation and tissue damage with injections.” Chapter 2 in Injectable Drug Development, eds. P. K. Gupta and G. A. Brazeau. Interpharm, 1999). Because inflammation can be severely damaging, it is particularly useful to control the underlying inflammatory reaction that often accompanies parenteral administration of drugs, rather than solely focus on a single symptom, such as pain. Thus, as shown in the application examples, in one preferred embodiment, the methods and compositions of the invention comprising microparticles of an analgesic agent surprisingly and beneficially can be used to prevent or mitigate symptoms of inflammation including, but not limited to, erythema, swelling, leukocyte migration, necrosis, endothelial loss, thrombosis, edema, and hemorrhage in addition to reducing injection site pain.

Furthermore, the aforementioned reduction in pain and/or inflammation achieved in the application examples corroborates that the methods and compositions of the invention can be used to prevent or reduce immunological reactions. Thus, although the invention is largely focused on the pain and inflammation associated with parenteral administration of a primary therapeutic agent, the applicants note that the observations in the application Examples demonstrating that parenterally administering microparticles of analgesic agent can surprisingly and beneficially reduce both the pain and inflammation associated with an insult (such as that caused by parenteral administration of a primary therapeutic) corroborate and indicate that parenteral administration of microparticles of an analgesic agent can also be used to treat an adverse immunological response. Pain, inflammation, and adverse immunological responses generally are mediated by the same immune responses. In this respect, it is known that parenterally administering a primary therapeutic agent to a subject in need thereof can render the subject susceptible to adverse antigenic responses. In particular, the methods according to the invention can be used to treat immunological reactions such as those caused by primary therapeutic agents that render the subject susceptible to an adverse antigenic response, for example, proteins and peptides known to induce adverse antigenic responses in substantial populations, primary therapeutics formulated as compositions having a pH greater than 10 or having a pH less than 4.2, primary therapeutics formulated as compositions having an osmolality greater than 800 mosmol/L (including greater than 1 osmol/L) or having an osmolality less than 150 mosmol/L (including less than 100 mosmol/L), and/or primary therapeutics formulated as compositions including relatively toxic excipients such as polyethoxylated caster oil that can induce an allergic response. For example, the primary therapeutic agent may be a peptide or protein capable of eliciting a hypersensitivity, i.e., allergic, response. Co-administration of a dispersion comprising microparticles of an analgesic agent according to the invention can advantageously decrease the adverse antigenic response and other immunological reactions associated with parenteral administration of the primary therapeutic agent.

In one aspect, the primary therapeutic agent and microparticles of an analgesic agent may be administered in sustained release formulations with similar release profiles, such that the duration of action of both the primary therapeutic agent and the analgesic agent are substantially the same. In another aspect, the release of microparticles of an analgesic agent from a sustained release formulation can occur for a length of time greater than the period in which the primary therapeutic agent is released, thus providing extended relief from pain, irritation, and/or immunological reaction associated with parenteral administration of the primary therapeutic agent.

The reduction in the adverse and potentially dangerous effects of parenteral administration achieved using the invention minimizes the drawbacks of a parenterally administered therapeutic agent and allows for the development of formulations focused on maximizing efficacy, thereby increasing the overall utility of the therapeutic agent. Further, because of the aforementioned reduction in adverse and potentially dangerous effects, the methods according to the invention advantageously facilitate parenteral administration of particulate formulations of primary therapeutic agents. In one embodiment, a method according to the invention comprises parenterally administering to a subject in need thereof a therapeutically effective amount of a dispersion comprising microparticles of a primary therapeutic agent, the dispersion further comprising microparticles of an analgesic agent in an amount effective to reduce the pain, inflammation, and/or immunological reaction associated with parenterally administering the primary therapeutic agent, wherein the microparticles of the primary therapeutic agent and the microparticles of the analgesic agent have an effective particle size of less than 20 micrometers.

In another embodiment, a method according to the invention comprises parenterally co-administering to a subject in need thereof a therapeutically effective amount of a first dispersion comprising microparticles of a primary therapeutic agent and a second dispersion comprising microparticles of an analgesic agent, wherein the second dispersion is administered in an amount effective to reduce the pain, inflammation, and/or immunological reaction associated with parenterally administering the primary therapeutic agent, wherein the microparticles of the primary therapeutic agent and the microparticles of the analgesic agent have an effective particle size of less than 20 micrometers.

In another embodiment, a method according to the invention comprises parenterally co-administering to a subject in need thereof a therapeutically effective amount of the primary therapeutic agent and a dispersion comprising microparticles of an analgesic agent, wherein the dispersion comprising microparticles of an analgesic agent is administered in an amount effective to reduce the pain, inflammation, and/or immunological reaction associated with parenterally administering the primary therapeutic agent, wherein the microparticles of the analgesic agent have an effective particle size of less than 20 micrometers.

In one embodiment, a pharmaceutical composition according to the invention comprises a dispersion comprising microparticles of an analgesic agent in an amount effective to reduce the pain, inflammation, and/or immunological reaction associated with parenterally administering a therapeutically effective amount of a primary therapeutic agent, wherein the microparticles of the analgesic agent have an effective particle size of less than 20 micrometers. In one aspect, the pharmaceutical composition further comprises a therapeutically effective amount of microparticles of a primary therapeutic agent, wherein the microparticles of the primary therapeutic agent have an effective particle size of less than 20 micrometers.

In another embodiment, a pharmaceutical composition according to the invention comprises a fibrin matrix and microparticles of an analgesic agent, said microparticles being dispersed within the fibrin matrix, wherein the microparticles of the analgesic agent have an effective particle size of less than 20 micrometers. In one aspect, the pharmaceutical composition is formed by mixing the microparticles of the analgesic agent with fibrinogen and then adding thrombin to the mixture to form a fibrin matrix containing dispersed microparticles. An example of a commercially available fibrin matrix composition is Tisseel® (Baxter International, Inc.).

In another embodiment, a method of preventing or reducing pain, inflammation, and/or immunological reactions in a subject suffering from arthritis according to the invention comprises delivering a pharmaceutical composition comprising a fibrin matrix and microparticles of an analgesic agent, optionally further comprising microparticles of a primary therapeutic agent, proximate to a site of arthritis, said composition being capable of releasing the analgesic agent and/or primary therapeutic agent in an amount effective for preventing or reducing pain, inflammation, and/or immunological reactions at the site of arthritis. In one aspect, the pharmaceutical composition is delivered directly to a site of arthritis, i.e., a joint, e.g., of the hand, wrist, elbow, jaw, neck, foot, shoulder, spine, ankle, hip, and/or knee. In another aspect, the pharmaceutical composition is delivered to the tissue and/or interstitial space surrounding or near the site of arthritis.

In a further embodiment, a method of preventing or reducing pain, inflammation, and/or immunological reactions at a site of surgery or at a wound site in a subject in need thereof according to the invention comprises delivering a pharmaceutical composition comprising a fibrin matrix and microparticles of an analgesic agent, optionally further comprising microparticles of a primary therapeutic agent, proximate to the site of surgery or the wound site, said composition being capable of releasing the analgesic agent and/or primary therapeutic agent in an amount effective for preventing or reducing pain, inflammation, and/or immunological reactions at the site of surgery or the wound site. In one aspect, the pharmaceutical composition is delivered directly to a site of surgery or at a wound site, e.g., at an incision, abrasion, contusion, laceration, and/or puncture. In another aspect, the pharmaceutical composition is delivered to the tissue and/or interstitial space surrounding or near said site of surgery or wound site. In various aspects of the foregoing embodiments, the analgesic agent may comprise ropivacaine. The ropivacaine may be substantially free of the (R)-isomer of ropivacaine. In another aspect, the pharmaceutical composition according to this embodiment of the invention further comprises microparticles of a primary therapeutic agent, said microparticles of the primary therapeutic agent being dispersed within the fibrin matrix, wherein the microparticles of the primary therapeutic agent have an effective particle size of less than 20 micrometers. In various aspects, the microparticles of the analgesic agent and/or primary therapeutic dispersed within the fibrin matrix have an effective particle size of less than 20 micrometers, less than 15 micrometers, less than 10 micrometers, less than 5 micrometers, or less than 3 micrometers. In one aspect, the microparticles of the analgesic agent and/or the microparticles of the primary therapeutic agent are released from the fibrin matrix over a course of at least one day, at least two days, at least three days, at least four days, at least five days, at least six days, at least one week, or more.

The following definitions may be useful in aiding the skilled practitioner in understanding the invention:

As used herein, an “adverse antigenic response” is an undesired immunological reaction triggered by an antigen. Adverse antigenic responses include four types of hypersensitivity reactions: 1) immediate, mediated primarily by IgE in response to antigens; 2) cytotoxic, mediated primarily by IgM or IgG and complement; 3) immune complex, mediated primarily by IgG and complement; and 4) delayed-type, mediated primarily by T-cells.

As used herein, an “analgesic agent” is a drug administered to a subject to prevent or relieve pain.

As used herein, a “depot” is an injected or implanted pharmaceutical formulation containing a reservoir of therapeutic agent and/or an analgesic agent that releases a therapeutically effective amount of the agent over an extended period of time, e.g., days or weeks.

As used herein, a “dispersion” is a mixture having at least one dispersed or discontinuous phase present in a solid, semi-solid or non-solid continuous phase. Representative examples of dispersions in accordance with the disclosure include, but are not limited to, solid-in-solid, solid-in-liquid, solid in gas (including solid in liquid in gas) compositions. A dispersion can be substantially homogenous or non-homogenous. A suspension is a particular dispersion in which the discontinuous solid phase, e.g., microparticles, can remain stably suspended, i.e., substantially free of aggregation, in the continuous phase for any extended period of time, e.g., days or weeks.

As used herein, a “microparticle” is a solid or semi-solid particle having an effective particle size less than 20 micrometers as measured by, for example, dynamic light scattering methods such as photocorrelation spectroscopy, laser diffraction, low-angle laser light scattering (LALLS), medium-angle laser light scattering (MALLS), light obscuration methods such as the Coulter method, rheology, or light/election microscopy. Microparticles can be amorphous, semicrystalline, crystalline, or a combination thereof as determined by suitable analytical methods such as differential scanning calorimetry (DSC) or X-ray diffraction.

As used herein, an “immunological reaction” refers to a physiological response to parenteral administration of a primary therapeutic agent that is mediated by a body's immune system. Immunological reactions include autoimmune disorders, and hypersensitivity reactions.

As used herein, a “matrix” is a three-dimensional composition formed from a material, typically a network of synthetic and/or naturally-occurring polymers, capable of containing and releasing a primary therapeutic agent and/or an analgesic agent over an extended period of time, e.g., days or weeks. A “fibrin matrix” refers to a three-dimensional composition comprising fibrin, a protein which can be obtained as the reaction product of fibrinogen and thrombin.

As used herein, the terms “parenteral” and “parenterally” refer to the administration of an agent via any route other than oral administration. For example, parenteral administration may comprise injection, infusion, implantation or any other mode of delivery other than ingestion to any site in or on the body of a subject.

As used herein, a “primary therapeutic agent” is an agent administered to a subject in need thereof that is capable of preventing, reducing, treating, and/or ameliorating the symptoms, pathology, and/or progression of a condition or disease affecting the subject.

As used herein, “proximate” refers to a location at, adjacent to, or near a reference site. For example, delivery of a pharmaceutical composition proximate to a site of arthritis, site of surgery, or a wound site refers to delivery of the composition directly to said site, as well as delivery of the composition to tissue and/or interstitial space contacting, surrounding, or near to said site.

As used herein, a “subject” is a non-plant, non-protist living being. In one aspect, the subject is an animal. In particular aspects, the animal is a mammal. In more particular aspects, the mammal is a human. In other aspects, the mammal is non-human, such as a rodent, cat, dog, horse, or cow. As used herein, a “subject in need thereof” is a subject suffering from a condition or disease who would benefit from the administration of a primary therapeutic agent and/or analgesic agent.

As used herein, the term “composition comprising the (S)-isomer” refers to a composition of a drug having a single stereocenter or a pharmaceutically acceptable salt thereof which is substantially free of the (R)-isomer of the drug or a pharmaceutically acceptable salt thereof. The term “substantially free of the (R)-isomer” refers to a composition containing less than 10% by weight, less than 5% by weight, less than 3% by weight, less than 2% by weight, less than 1% by weight, and/or less than 0.5% by weight of the (R)-isomer of the drug based on the total amount of drug in the composition. The total (R)-isomer and (S)-isomer content can be determined using a standard HPLC column or other analytical methods known in the art.

As used herein, the term “sustained release” refers to the release of a primary therapeutic agent and/or an analgesic agent from a formulation in a way that deviates from immediate release, i.e., less than 50% of the agent is released in the first 30 minutes, the first 90 minutes, the first 24 hours, and/or the first seven days following administration. Thus, sustained release includes release of an agent from a formulation for an extended period of time, e.g., hours, days, and/or weeks. In one exemplary embodiment, sustained release refers to a formulation which releases 100% of the analgesic agent in 24 hours, 36 hours, 48 hours, or 60 hours, which formulation provides a persistent therapeutic effect for 3-10 days, for example 7 days, after release is complete. In another exemplary embodiment, sustained release refers to a formulation which releases 100% of the primary therapeutic agent and/or the analgesic agent over 30 days or 1 month time. Such sustained release formulations are particularly preferred by both clinicians and recipients in that administration of the primary therapeutic and/or the analgesic agents does not have to be accomplished as regularly. Most preferably, the sustained release period of the primary therapeutic agent and the analgesic agent is substantially the same, e.g., differing only by 2-3 days or less.

The terms “therapeutically effective amount,” “effective amount,” and “amount effective” are used synonymously and refer to the amount of a primary therapeutic agent and/or analgesic agent necessary to achieve a desired therapeutic result in a subject. For example, in certain aspects of the invention, a therapeutically effective amount of a primary therapeutic agent would be the amount necessary to reduce and/or ameliorate the symptoms associated with a disease or disorder. An effective amount of an analgesic agent can be the amount necessary to prevent or reduce pain, inflammation, and/or immunological reactions associated with parenteral administration of a therapeutic agent. Alternatively, an effective amount of an analgesic agent can be the amount necessary to prevent or reduce pain, inflammation, and/or immunological reactions associated with arthritis, a wound site, a site of surgery, or a site of pain. Of course, one of ordinary skill in the art understands that the “therapeutically effective amount,” “effective amount,” and “amount effective” of a primary therapeutic agent and/or an analgesic agent will depend upon the therapeutic context and objectives. Additionally, therapeutically effective amounts of the primary therapeutic agent and the analgesic agent administered are based on subject parameters such as the weight and condition of the subject and can be easily determined by the skilled practitioner using known dosing protocol information which can be adjusted as needed in view of ascertainable formulation variables such as water solubility, particle size, and total amount of drug in a given dose. See, for example, Turco, “Sterile Dosage Forms” 4^{th} Ed., Lea & Febiger, 1994. Further considerations relating to determining an appropriate “therapeutically effective amount” are known to the skilled clinician and described, in part, below.

The methods and pharmaceutical compositions according to the invention comprise a dispersion comprising microparticles of an analgesic agent and may further include a dispersion comprising microparticles of a primary therapeutic agent. In one aspect, the microparticles of the primary therapeutic agent and/or the microparticles of the analgesic agent have an effective particle size greater than 100 nanometers and less than 20 micrometers. For example, the effective particle size may be greater than 100 nanometers and less than 15 micrometers, the effective particle size may be greater than 100 nanometers and less than 10 micrometers, the effective particle size may be greater than 100 nanometers and less than 5 micrometers, the effective particle size may be greater than 100 nanometers and less than 1 micrometer, the effective particle size may be greater than 100 nanometers and less than 400 nanometers, the effective particle size may be greater than 100 nanometers and less than 200 nanometers, and/or the effective particle size may be greater than 100 nanometers and less than 150 nanometers. As a result, the term “nanoparticle” is encompassed by the term “microparticle” as defined herein. The processes for preparing the microparticles used in the present invention can be accomplished through numerous techniques known in the art. A representative, but non-limiting discussion of techniques for preparing microparticles follows.

Energy addition methods generally involve adding a pharmaceutically active compound in bulk form to a suitable vehicle such as water or aqueous solution. The vehicle typically contains one or more of the surfactants set forth below or any other liquid in which the pharmaceutical compound is not appreciably soluble, to form a first suspension that can be referred to as a presuspension. Energy is added to the presuspension to form a particle dispersion which is physically more stable than the presuspension. Energy is added by mechanical grinding, e.g., pearl milling, ball milling, hammer milling, fluid energy milling, jet milling, or wet milling. The presuspension may be further subjected to high shear conditions including cavitation, shearing, or impact forces utilizing a microfluidizer. Energy can also be added to the presuspension using a homogenizer such as a piston gap homogenizer or counter current flow homogenizer. The addition of energy can also be accomplished using sonication techniques carried out using any suitable sonication device. Typically, the sonication device has a sonication horn or probe that can be inserted into the presuspension to emit sonic energy into the solution. Examples of such techniques are disclosed in U.S. Pat. Nos. 5,145,684 and 5,091,188.

Microprecipitation methods generally involve dissolving an organic compound in a water-miscible first organic solvent to create a first solution and then mixing the first solution with a second solvent or water to precipitate the organic compound to create a presuspension. Energy can then be added to the presuspension as discussed above to form microparticles. For example, a tandem microprecipitation-homogenization method can be used to obtain a microparticle dispersion. Optionally, the first organic solvent is removed from the mixture by any suitable means such as centrifugation or filtration methods. One or more optional surfactants set forth below can be added to the first organic solvent, to the second aqueous solution, or to both the first organic solvent and the second aqueous solution. Examples of microprecipitation processes are disclosed in U.S. Pat. Nos. 5,780,062, 6,607,784, 6,869,617, 6,884,436, and 7,037,528.

Emulsion precipitation methods generally involve providing a multiphase system having an organic phase containing a pharmaceutically active compound and an aqueous phase, the organic phase having the pharmaceutically active compound therein, and sonicating the system to evaporate a portion of the organic phase to cause precipitation of the compound in the aqueous phase to form a dispersion of microparticles. The microparticle dispersion can optionally be lyophilized. The step of providing a multiphase system includes (1) mixing a water-immiscible solvent with a pharmaceutically active compound to define an organic solution; (2) preparing an aqueous-based solution with one or more surface active compounds; and (3) mixing the organic solution with the aqueous solution to form the multiphase system. The organic and aqueous phases can be mixed using homogenizers, colloidal mills, high speed stirring equipment, extrusion equipment, manual agitation or shaking equipment, a microfluidizer, or other equipment or techniques for providing high shear conditions. The crude emulsion will have oil droplets in water that are approximately less than one micrometer in diameter. The crude emulsion can be sonicated to define a microemulsion and eventually to provide a dispersion of microparticles. Examples of emulsion precipitation methods are disclosed in U.S. Patent Pub. No. 2005/0037083 and U.S. Pat. No. 6,835,396.

Solvent-antisolvent precipitation methods generally involve a dispersion created by (1) preparing a liquid phase of an active substance in a solvent or a mixture of solvents which may contain one or more surfactants; (2) preparing a second liquid phase of a non-solvent or a mixture of non-solvents miscible with the preparation from (1); (3) adding together the solutions of (1) and (2) with stirring; and (4) removing unwanted solvents to produce a dispersion of microparticles. Unlike the methods described above, a final step of adding energy to the suspension to form the dispersion is not necessary. Examples of solvent-antisolvent precipitation techniques are disclosed in U.S. Pat. Nos. 5,118,528 and 5,100,591.

Other methods for producing dispersions comprising microparticles that may be used in accordance with the invention include, but are not limited to, phase inversion precipitation, pH shift precipitation, infusion precipitation, temperature shift precipitation, solvent evaporation precipitation, reaction precipitation, compressed fluid precipitation, spraying onto cryogenic fluids, and protein microsphere precipitation.

Microparticle dispersions can be formed using one or more surfactants. Suitable surfactants may be anionic, cationic, zwitterionic and/or nonionic surfactants. Examples of surfactants include, but are not limited to, alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl glycerol, phosphatidyl inosine, phosphatidylinositol, diphosphatidylglycerol, phosphatidylserine, phosphatidic acid and their salts, sodium carboxymethylcellulose, cholic acid and other bile acids, phosphatidylcholine, phosphatidylethanolamine, diacyl-glycero-phosphoethanolamine, dimyristoyl-glycero-phosphoethanolamine (DMPE), dipalmitoyl-glycero-phosphoethanolamine (DPPE), distearoyl-glycero-phosphoethanolamine (DSPE), distearoyl-phosphatidyl-ethanolamine-methyl-polyethyleneglycol conjugate (mPEG-DSPE), dioleolyl-glycero-phosphoethanolamine (DOPE), polyethylene glycol (PEG), benzalkonium chloride, cetyltrimethylammonium bromide, chitosans, lauryldimethylbenzylammonium chloride, acyl carnitine hydrochlorides, dimethyldioctadecylammomium bromide (DDAB), dioleoyltrimethylammonium propane (DOTAP), N-[1-(2,3-dioleyloxy)propyl]-N,N,N-trimethylammonium (DOTMA), dimyristoyltrimethylammonium propane (DMTAP), dimethylaminoethanecarbamoyl cholesterol (DC-Chol), 1,2-diacylglycero-3-(O-alkyl)phosphocholine, O-alkylphosphatidylcholine, alkyl pyridinium halides, long-chain alkyl amines, n-octylamine and oleylamine glyceryl esters, polyoxyethylene fatty alcohol ethers, polyoxyethylene sorbitan fatty acid esters (polysorbates), polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethylcellulose, hydroxypropylcellulose, hydroxypropylmethylcellulose, noncrystalline cellulose, polysaccharides including starch and starch derivatives such as hydroxyethylstarch (HES), polyvinyl alcohol, and polyvinylpyrrolidone.

In one aspect, a primary therapeutic agent according to the invention comprises a drug, diagnostic agent, or vaccine associated with pain on injection. The primary therapeutic agent can be selected from a variety of known pharmaceutical compounds including, but not limited to: analeptics, anti-cancer agents, antibodies, adrenergic agents, adrenergic blocking agents, adrenolytics, adrenomimetics, anti-cholinergic agents, anti-cholinesterases, anticonvulsants, alkylating agents, alkaloids, allosteric inhibitors, anorexiants, antacids, anti-diarrheals, antidotes, anti-folics, anti-pyretics, anti-rheumatic agents, psychotherapeutic agents, anti-helmintics, anti-coagulants, anti-depressants, anti-epileptics, anti-fibrotic agents, anti-infective agents (e.g., anti-fungals, antibiotics, and anti-viral agents including anti-retroviral agents such as protease inhibitors, nucleoside reverse transcriptase inhibitors, non-nucleoside reverse transcriptase inhibitors, entry inhibitors which are also called fusion inhibitors, and integrase inhibitors), antihistamines, anti-muscarinic agents, anti-mycobacterial agents, anti-neoplastic agents, anti-protozoal agents, anxiolytics, beta-adrenoceptor blocking agents, cough suppressants, dopaminergics, hemostatics, hematological agents, hypnotics, immunological agents, muscarinics, parasympathomimetics, peptides, proteins, prostaglandins, radio-pharmaceuticals, stimulants, sympathomimetics, vitamins, xanthines, vaccines, growth factors, hormones, antiprion agents, diagnostic agents, and combinations thereof. A description of classes of therapeutic agents and a listing of species within each class can be found in Martindale, The Extra Pharmacopoeia, 31st Edition, The Pharmaceutical Press, London, 1996. The listed therapeutic agents are commercially available and/or can be prepared by known techniques. In one aspect, a primary therapeutic agent according to the invention is selected from the group consisting of peptides, proteins, antibodies, anti-retroviral drugs, psychotherapeutic agents, bisphosphonates, and combinations thereof. In one aspect, the primary therapeutic agent and the analgesic agent are different both structurally and functionally, i.e., the primary therapeutic agent and the analgesic agent not only comprise different compounds but are also members of different therapeutic classes. Accordingly, in a preferred aspect, the primary therapeutic is not a drug selected from the group consisting of antihistamines, mast cell stabilizers, corticosteroids, anti-inflammatories, local anesthetics, and combinations thereof, i.e., the primary therapeutic is not an analgesic agent according to the invention.

Exemplary primary therapeutic agents that may cause irritation when administered parenterally include, but are not limited to, abciximab, abobotulinumtoxina, adalimumab, aminocaproic acid, anakinra, anti-inhibitor coagulant complex, anti-hemophilic factor, aprepitant, arformoterol tartrate, bisphosphonates, bortezomib, botulinum toxin types A and B, calcitriol, certolizumab pegol, chloramphenicol palmitate, chloramphenicol sodium succinate, choriogonadotropin alfa, chorionic gonadotrophin, cilastatin, coagulation Factor VIIa, dalteparin sodium, darbepoetin alfa, decitabine, dexrazoxane hydrochloride, digoxin, follitropin alpha, follitropin beta, fosphenyloin, fulvestrant, enoxaparin sodium, epopostenol sodium, ertapenem, esmolol, estrogens, etonogestrel, glatiramer acetate, human immune globulin intravenous, ibandronate, imipenem, interferon alfa-2b, interferon beta-1b, insulin glargine, interferon gamma-1B, lacosamide, maraviroc, mitomycin, onabotulinumtoxina, octreotide, arsenic trioxide, olanzapine, ondansetron, peginterferon alfa-2b, phenyloin, piperacillin, rocuronium bromide, sodium hyaluronate, tazobactam, teriparatide, tigecycline, risperidone, ziprasidone hydrochloride, ziprasidone mesylate, zoledronic acid, azithromycin, bivalirudin, busulfan, carboprost tromethamine, cytarabine, danaparoid, dimercaprol, divalproex, doxorubicin, ferric gluconate, foscarnet, furosemide, gadobenate dimeglumine, gadobutrol, gadofosveset, gadoteridol, hydroxocobalamin, incobotulinumtoxin A, interferon alfacon-1, interferon beta-1a, iopromide, ioversol, laronidase, leuprolide, meropenem, mesna, naltrexone, nicardipine, ribavirin phentolamine, rimabotulinumtoxin B, somatropin, thiotepa, treprostinil, triptorelin, valproic acid, valproic acid derivatives, varicella-zoster immunoglobulin, vinorelbine, cytarabine, alefacept, bivalirudin, edetate calcium disodium, epirubicin hydrochloride, flumazenil, idarubicin hydrochloride, lansoprazole, mitoxantrone hydrochloride, paromomycin sulfate, polymyxin B sulfate, pralidoxime chloride, progesterone, alpha-1-proteinase inhibitor, dipyridamole, epirubicin hydrochloride, epoprostenol sodium, valproate sodium, tobramycin, cefotetan, desmopressin, doxycycline, atropine, atropine sulfate dimenhydrinate, edetate calcium disodium, penicillin G, promethazine hydrochloride, spectinomycin hydrochloride, aminoglycosides, aminopenicillins, ampicillin sodium, sulbactam sodium, cardiac glycosides, ceftazidime, labetalol hydrochloride, methocarbamol, pralidoxime chloride, spectinomycin hydrochloride, terbutaline sulfate, amidotrizoic acid, bleomycin sulfate, carbenicillin sodium, cefalotin sodium, clarithromycin, clodronate, clodronic acid, dacarbazine, epoetin alfa, epoetin beta, epoetin delta, epoetin gamma, epoetin omega, epoetin theta, epoetin zeta, meglumine amidotrizoate, oxamniquine, sermorelin acetate, sodium amidotrizoate, somatorelin, sumatriptan, sumatriptan succinate, vesnarinone, tobramycin, thymostimulin, thymopentin, teceleukin, pidotimod, oprelvekin, interleukins, simvastatin, triflusal, denileukin diftitox, celmoleukin, aldesleukin, trivax, nadolol, INGAP, ilodecakin, denenicokin, darleukin, losartan, oncostatin M, and combinations thereof. Exemplary vaccines associated with injection site pain include, but are not limited to, vaccines against diphtheria, influenza, Haemophilus influenzae type B, Hepatitis A, Hepatitis B, human papillomavirus, measles, meningitis, mumps, pertussis, polio, rabies, rubella, tetanus, and combinations thereof.

In one aspect, the primary therapeutic agent comprises an anti-retroviral drug, for example, an anti-HIV drug. In one aspect, the primary therapeutic agent may comprise a drug selected from the group consisting of protease inhibitors, nucleoside reverse transcriptase inhibitors, non-nucleoside reverse transcriptase inhibitors, entry inhibitors which are also called fusion inhibitors, integrase inhibitors, and combinations thereof. Examples of protease inhibitors include, but are not limited to, fosamprenavir, indinavir, ritonavir, saquinavir, nelfinavir, atazanavir and combinations thereof, such as a combination of ritonavir and atazanavir. Examples of nucleoside reverse transcriptase inhibitors include, but are not limited to, abacavir, zidovudine, didanosine, stavudine, zalcitabine, lamivudine and combinations thereof. Examples of non-nucleoside reverse transcriptase inhibitors include, but are not limited to, efavirenz, nevirapine, delaviradine (mesylate), and combinations thereof. Examples of fusion inhibitors include, but are not limited to, maraviroc and enfuvirtide. Examples of integrase inhibitors include, but are not limited to, dolutegravir and S/GSK1265744 (ViiV Healthcare). Exemplary combinations of anti-retroviral drugs include, but are not limited to, ritonavir/atazanavir/efavirenz, lamivudine/zidovudine, lamivudine/abacavir, abacavir/lamivudine/zidovudine, and dolutegravir/lamivudine/abacavir. Typical combinations include two nucleoside reverse transcriptase inhibitors plus one protease inhibitor or two nucleoside reverse transcriptase inhibitors plus one non-nucleoside reverse transcriptase inhibitors.

An analgesic agent according to the invention may comprise any analgesic agent known in the art. A dispersion comprising microparticles of an analgesic agent according to the invention can advantageously prolong the analgesic agent's duration of action, compared to a liquid formulation, and allow for increased drug loading, e.g., in weight per unit volume of tissue, which can also extend the active period of the compound. In one aspect, the analgesic agent comprises a drug selected from the group consisting of antihistamines, mast cell stabilizers, corticosteroids, anti-inflammatories including but not limited to Substance P inhibitors and IL-18 inhibitors, local anesthetics, and combinations thereof. In certain aspects, the invention includes amino amide anesthetics, amino ester anesthetics, amino amide derivatives, and their salts, hydrates, and prodrugs. Examples of suitable analgesic agents include, but are not limited to, local anesthetic agents such as lidocaine, mepivacaine, prilocalne, etidocaine, bupivacaine, levobupivacaine, ropivacaine, dibucaine, articaine, cocaine, procaine, mepivacaine, prilocalne, articaine, benzocaine, chloroprocaine, etidocaine, tetracaine, dibucaine, butamben, capsaicin, their salts, hydrates, prodrugs, and combinations thereof.

In one aspect, the analgesic agent is an inhibitor of the proinflammatory agents Substance P and/or Interleukin-18 (IL-18). Substance P is a neuropeptide that orchestrates the inflammatory response by eliciting ingress of inflammatory white cells into the tissue area. IL-18 is a cytokine that induces natural killer and T cells to release interferon. The inhibition of Substance P and/or IL-18 can be measured, for example, by analyzing tissue samples to determine the concentrations of the agents and downstream cytokines. Inhibiting mediators of the inflammatory cascade such as Substance P and IL-18 reduces inflammation and its symptoms, resulting in more effective and prolonged pain relief. Ropivacaine, lepobupivacaine, and lidocaine can all act as inhibitors of Substance P (Dias et al., Anaesthesia, 2008; 63(2):151-5). Other examples of inhibitors of Substance P and/or IL-18 include, but are not limited to ustekinumab, tocilizumab, sareito, tacrolimus, rilonacept, iguratimod, hydrocortisone, diacerein, aceclofenac, daclizumab, canakinumab, basiliximab, actarit, sirukumab, secukinumab, sarilumab, reslizumab, reparixin, MK-3222 (Merck), mepolizumab, MABp1 (XBiotech), lebrikizumab, ixekizumab, inolimomab, gevokizumab, brodalumab, briakinumab, tralokinumab, iltuximab, olokizumab, NN-8226 (Novo Nordisk), lisofylline, guselkumab, GSK-1070806 (GlaxoSmithKline), givinostat, dupilumab, dersalazine sodium, clazakizumab, benralizumab, ASM-8 (Pharmaxis), anrukinzumab, AN-2898 and AN-2728 (Anacor), AMG-139 and AMG-108 (Amgen), ALX-0061 (Ablynx), AC-201 (TWi Biotechnology), TT-301 and TT 302 (Transition Therapeutics), SA-237 (Chugai), NI-1401 and NI-1201 (Novimmune), MEDI-5117 (AstraZeneca), HMPL-011 (Hutchison MediPharma), EBI-005 (Eleven Biotherapeutics), BMS-981164 (Bristol-Myers Squibb), BI-655066 (Boehringer Ingelheim), ABT-981 and ABT-122 (Abbott), XT-101 (Xalud Therapeutics), SM-401 (SuppreMol), ralfinamide, PRS-060 (Pieris), inflammasome modulators, IL-6 inhibitors, IL-6 antagonists, IL-15 antagonist, IL-12/23 inhibitors, HuMax-IL8 (Cormorant), E-36041 (Ensemble Therapeutics), DRM-02 (Dermira), ARGX-109 (arGEN-X), and combinations thereof. Further still, suitable inhibitors of Substance P and/or IL-18 can be identified using the assay described in the application examples.

In one aspect, the primary therapeutic agent and/or the analgesic agent, most typically the analgesic agent, is a poorly water-soluble compound, i.e., the solubility of the compound in water is less than about 10 mg/mL, and preferably less than about 1 mg/mL, for example, less than 0.5 mg/mL. These poorly water-soluble compounds are particularly suitable for aqueous suspension preparations since there are limited alternatives for formulating these compounds in an aqueous medium. Surfactants can adsorb to the surface of particles comprising such poorly water soluble active agents to form a substantially uniform coating thereon. For example, the hydrophobic tail moieties of surfactants can associate with hydrophobic regions on the particle surface. In addition, electrostatic interactions between the surfactant and negatively charged regions on the particle surface can stabilize the coating comprising the surfactant. Such surfactant coatings can advantageously increase the stability of a dispersion such that particle aggregation is substantially reduced.

Alternatively, the primary therapeutic agent and/or analgesic agent can be a water-soluble compound. To form aqueous suspensions of water-soluble compounds the water soluble active compounds can be entrapped in a solid carrier matrix (for example, polylactate-polyglycolate copolymer, albumin, or starch) or encapsulated in a surrounding vesicle that is substantially impermeable to the active agent. An encapsulating vesicle can be a polymeric coating such as polyacrylate. Further, the microparticles containing these water soluble compounds can be modified to improve chemical stability and control the pharmacokinetic properties of the compounds, for example, by controlling the release of the compounds from the microparticles. Examples of water-soluble compounds include, but are not limited to, simple organic compounds, proteins, peptides, nucleotides, and carbohydrates.

In one aspect, the analgesic agent comprises the (S)-isomer of ropivacaine and/or bupivacaine and/or their salts and/or prodrugs. In one embodiment, the analgesic agent comprising the (S)-isomer of ropivacaine and/or bupivacaine and/or their salts and/or prodrugs is substantially free of the (R)-isomer form. For example, the analgesic agent comprising the (S)-isomer of ropivacaine and/or bupivacaine contains the (R)-isomer of ropivacaine and/or bupivacaine in an amount less than 10% by weight, less than 5% by weight, less than 3% by weight, less than 2% by weight, less than 1% by weight, and/or less than 0.5% by weight of the drug or a pharmaceutically acceptable salt thereof. In a particular aspect, the analgesic agent comprises ropivacaine, a ropivacaine salt, a ropivacaine prodrug, a ropivacaine analog, a ropivacaine derivative, or a combination thereof.

The primary therapeutic agent and the analgesic agent according to the invention may be administered simultaneously. In one aspect, the primary therapeutic agent and analgesic agent are administered concurrently in a single dispersion or pharmaceutical composition containing both the primary therapeutic agent and the microparticles of the analgesic agent. Alternatively, the primary therapeutic agent may be administered separately; for example, the primary therapeutic agent may be administered before the analgesic agent or the analgesic agent may be administered before the primary therapeutic agent.

The primary therapeutic agent may be parenterally administered according to the invention in a number of formulations, such as microparticle dispersions, solutions, emulsions, liposomes, implants, and combinations thereof. In aspects of the invention, the primary therapeutic agent and the microparticles of the analgesic agent can be parenterally administered to a subject through varied routes, most frequently by injection, infusion, or implantation. In some aspects, the primary therapeutic agent and the microparticles of the analgesic agent can be delivered via injection, for example, by intraarticular, intracerebral (intraparenchymal), intracerebroventricular, intracerebrospinal, intracranial, intramuscular, intradermal, intraperitoneal, subcutaneous, intraocular, intraportal, intranasal, or intralesional routes. Typically, the administration of the primary therapeutic agent and/or the microparticles of the analgesic agent is via intraarticular injection, intradermal injection, subcutaneous injections, and/or intramuscular injection.

In addition, the primary therapeutic agent and the microparticles of the analgesic agent can be introduced for treatment into a mammal by parenteral modes including, but not limited to, intratumor, topical, subconjunctival, intrabladder, intravaginal, epidural, intracostal, inhalation, transdermal, transserosal, intrabuccal, dissolution in the mouth or other body cavities, instillation to the airway, insufflation through the airway, injection into vessels, tumors, organ and the like, and injection or deposition into cavities in the body of a mammal. In a particular aspect, the primary therapeutic agent and/or the microparticles of the analgesic agent are delivered surgically, e.g., by implantation. In a further aspect, the primary therapeutic and/or the microparticles of the analgesic agent are delivered in a spray. A spray containing microparticles of the analgesic agent can advantageously be administered transdermally in the site of surgery (e.g., caused by implantation of a depot or other sustained release formulation of the primary therapeutic agent) or injury (e.g., caused by injection of the primary therapeutic agent).

Delivery of the primary therapeutic agent and/or the microparticles of the analgesic agent can be administered to any site in the body. In certain aspects, the site of administration is in the nerves, liver, kidney, heart, lung, eye, gastrointestinal tract, skin, and/or brain. In one aspect, the site of administration is any site of the body in need of pain prevention or pain relief.

The primary therapeutic agent and the microparticles of the analgesic agent may be delivered into a subject using a variety of different means. In one aspect, direct injection by needle and syringe can be used. In certain aspects, direct injection includes mixing microparticles of the primary therapeutic agent and the analgesic agent in the syringe immediately prior to administration or injection in a subject. In other aspects, the invention includes the use of a mixing chamber between the syringe and needle to facilitate mixing. In some aspects, the primary therapeutic agent and microparticles of the analgesic agent are administered by bolus injection or by implantation device. In certain aspects, a bolus injection is given by intravenous infusion or by direct injection, using a syringe. This mode of administration may be desirable in surgical patients, if appropriate, such as patients having cardiac surgery, e.g., coronary artery bypass graft surgery and/or valve replacement surgery. However, infusion and other continuous administration methods are generally disfavored because of the inconvenience and discomfort that the subject often experiences during administration. Thus, in one aspect, neither the primary therapeutic agent nor the analgesic agent is administered by a continuous administration method. In other aspects, a single injection is given intramuscularly or subcutaneously. Shorter or longer time periods of administration can be used, as determined to be appropriate by one of skill in this art.

Alternatively or additionally, the primary therapeutic agent and the microparticles of the analgesic agent are administered locally via implantation of a membrane, sponge, or another appropriate material onto/into which the dispersion has been absorbed or encapsulated. Where an implantation device is used, the device, in various aspects, is implanted into any suitable tissue or organ, and delivery of the primary therapeutic agent and/or the microparticles of the analgesic agent may be via diffusion, sustained release, bolus, or continuous release.

Separate dispersions, one containing microparticles of the primary therapeutic agent and the other containing microparticles of the analgesic agent, can be used. Alternatively, a single dispersion can contain microparticles of both the primary therapeutic agent and the analgesic agent.

In one aspect, the dispersion(s) comprising the primary therapeutic agent and/or analgesic agent is a sustained release formulation. Sustained release formulations known in the art that are suitable for use with the invention include but are not limited to depot injections, in situ forming implants, polymer matrices, tissue sealants, glues, and combinations thereof. Other specific examples of sustained release formulations include, but are not limited to, oil-based solutions, injectable drug suspensions, liposomes (e.g., DepoFoam® (Pacira Pharmaceuticals, Inc. Parsippany, N.J.), and polymer-based microspheres. Sustained release formulations can also be developed by altering microparticle size, using specific crystal forms and/or using hydrophobic salts. In one aspect, the sustained release formulation releases the primary therapeutic and the analgesic agent for substantially the same period of time. In another aspect, the half-life, i.e., the time needed to release half of the drug initially present in the formulation, of the formulation containing the analgesic agent is greater than the half-life of the formulation containing the primary therapeutic agent. In another aspect, the half-life of the formulation containing the analgesic agent is less than the half-life of the formulation containing the primary therapeutic agent, but the analgesic agent is still effective for reducing the pain, inflammation, and/or immunologic reaction associated with parenterally administering the primary therapeutic agent over the residence time of the primary therapeutic agent even when the analgesic agent is no longer detectable in the blood or even in a tissue of a subject. Polymers suitable for use in sustained release formulations include, but are not limited to, polylactides (PLA), polyglycolides (PGA), poly(lactide-co-glycolide) (PLGA), polycaprolactone (PCL), polyethylene glycol (PEG), polyglyconate, polypropylene glycol (PPG), polyanhydrides, polyorthoesters, polyhydroxybutyrate (PHB), poly(dioxanone), polyalkylcyanoacrylates, chitosan, and combinations thereof.

In another aspect, the primary therapeutic agent and/or the analgesic agent are administered in the form of a depot injection. Separate depot injections, one containing the primary therapeutic agent and the other containing microparticles of the analgesic agent, can be used. Alternatively, a single depot injection can contain both the primary therapeutic agent and the analgesic agent.

In another aspect, the primary therapeutic agent and/or the analgesic agent are administered in the form of an in situ forming implant, for example, as described in Kempe and Mader, J Control Release. 2012; 161(2): 668-79. Examples of in situ forming implants include, but are not limited to, thermoplastic pastes, in situ cross-linked polymer systems, in situ polymer precipitation, thermally induced gelling systems, and in situ solidifying organogels. Separate in situ forming implants, one containing the primary therapeutic agent and the other containing microparticles of the analgesic agent can be used. Alternatively, a single implant can contain microparticles of both the primary therapeutic agent and the analgesic agent.

In a further aspect, the primary therapeutic agent and/or the analgesic agent is incorporated in a matrix. For example, the microparticles of the analgesic agent can be incorporated in a matrix. In one embodiment, the matrix further comprises microparticles of the primary therapeutic. In another embodiment, separate matrices, one containing the primary therapeutic agent and the other containing microparticles of the analgesic agent, can be used. A matrix may be composed of natural polymers such as fibrinogen or collagen, synthetic polymers, or a combination thereof. Suitable synthetic polymers include, but are not limited to, polymers such as poly(lactide) (PLA), poly(glycolic acid) (PGA), poly(lactide-co-glycolide) (PLGA), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates, derivatives and combinations thereof.

In a further aspect, the primary therapeutic agent and/or the analgesic agent is incorporated into a tissue sealant/glue. For example, the microparticles of the analgesic agent can be incorporated into a tissue sealant/glue. In one embodiment, the tissue sealant/glue further comprises microparticles of the primary therapeutic. In another embodiment, separate tissue sealant/glues, one containing the primary therapeutic agent and the other containing microparticles of the analgesic agent, can be used. Tissue sealants are a type of surgical tissue adhesive used to control surgical bleeding, speed wound healing, close body organs or cover suture holes, and provide slow-release delivery of medications such as antibiotics to exposed tissues. Tissue sealants may comprise the natural and/or synthetic polymers listed above. Examples of commercially available tissue sealants include, but are not limited to, Tisseel® (Baxter International Inc.), BioGlue® (CryoLife), and TissuGlu® (Cohera Medical, Inc.).

In other aspects of the invention, additional ways of delivering the composition to a subject will be evident to those skilled in the art, including alternative formulations involving sustained release delivery.

One skilled in the art will appreciate that the appropriate therapeutically effective dosage levels for treatment will vary depending, in part, upon the tissue site to which the primary therapeutic agent and/or analgesic agent is delivered, the indication for which the treatment is being used, the route of administration, and the size (body weight, body surface or organ size) and condition (age and general health) of the patient. Accordingly, the clinician may adjust the dosage and modify the route of administration to obtain the optimal therapeutic effect.

Each publication, patent application, patent, and other reference cited herein is incorporated by reference in its entirety to the extent that it is not inconsistent with the present disclosure.

The following Examples are provided for illustration only and are not in any way to limit the scope of the invention.

### Example 1

### Microparticle Dispersions of an Analgesic Agent

Dispersions comprising microparticles of the analgesic agent ropivacaine were prepared using an energy addition/homogenization procedure. Five ropivacaine formulations were prepared using ropivacaine hydrochloride as starting material and are described in Table 1 below.

TABLE 1

| | Formulation | | | | | | | | | |
|---|---|---|---|---|---|---|---|---|---|---|
| Component | #1 | | #2 | | #3 | | #4 | | #5 | |
| Ropivacaine hydrochloride | 1% | (w/v) | 1% | (w/v) | 1% | (w/v) | 1% | (w/v) | 1% | (w/v) |
| Sodium phosphate | 0.3118 | g/L | 0.3118 | g/L | 0.3118 | g/L | 0.3118 | g/L | 0.3118 | g/L |
| monobasic, monohydrate | | | | | | | | | | |
| Sodium phosphate dibasic, | 1.0994 | g/L | 1.0994 | g/L | 1.0994 | g/L | 1.0994 | g/L | 1.0994 | g/L |
| anhydrous | | | | | | | | | | |
| Glycerin | 2.25% | (w/v) | 2.25% | (w/v) | 2.25% | (w/v) | 2.25% | (w/v) | 2.25% | (w/v) |
| Poloxamer 188 | 0.5% | (w/v) | — | | 0.5% | (w/v) | — | | 0.1% | (w/v) |
| DSPE-mPEG 2000 | 0.2% | (w/v) | — | | — | | — | | — | |
| Polysorbate 80 (Tween 80) | — | | — | | 0.25% | (w/v) | — | | — | |
| Lipoid E80 | — | | 1.2% | | — | | 1.2% | (w/v) | — | |
| Sodium | — | | — | | — | | — | | 0.1% | (w/v) |
| Deoxycholate | | | | | | | | | | |
| 1,2-Dimyristoyl-sn-glycero- | — | | — | | — | | 0.2% | (w/v) | — | |
| 3-phosphoglycerol, sodium | | | | | | | | | | |
| Water | QS | | QS | | QS | | QS | | QS | |
| Particle Size | 2 | μm | 6 | μm | 2 | μm | 3 | μm | 1.5 | μm |

To prepare the ropivacaine free base, 4 grams of ropivacaine HCl was added to 56 mL of water in a beaker. The solution in the beaker was stirred and heated at 75° C. to disperse and dissolve the drug. When the solution appeared visually clear, the heat was removed. Sodium hydroxide (NaOH, 1N) was added to the solution while stirring to cause precipitation. The addition of NaOH was continued until the solution pH was greater than 10. The solution was allowed to cool to room temperature and then filtered. The filtercake was dried in a vacuum oven. The resulting material was analyzed by differential scanning calorimetry (DSC). The melting point was found to be approximately 149° C., close to the published melting point of ropivacaine free base (144° C. to 146° C., Merck Index) and distinctly different from the melting point of ropivacaine hydrochloride (270° C., Merck Index).

Surfactant solutions were prepared by dissolving all the formulation components of Formulations #1 to #5 except ropivacaine in water. The pH of the solutions was approximately 7.4 to 7.5. Laboratory scale (20 mL) suspensions were prepared with a target concentration of 10 mg/mL ropivacaine using an energy addition/homogenization procedure. Briefly, ropivacaine free base was added to the surfactant solutions and dispersed using a rotor-stator mixer. The resulting dispersions were transferred to a piston-gap homogenizer and circulated at static pressure until the suspension temperature was less than 10° C. The dispersion was then homogenized at a target pressure of 20,000±2,000 psi and a target temperature of less than 10° C. Dispersions were collected after approximately 30 to 60 minutes of homogenization and had a final ropivacaine microparticle size of approximately 1.5 micrometers (Formulation #5), 2 micrometers (Formulation #1), 2 micrometers (Formulation #3), 3 micrometers (Formulation #4), or 6 micrometers (Formulation #2).

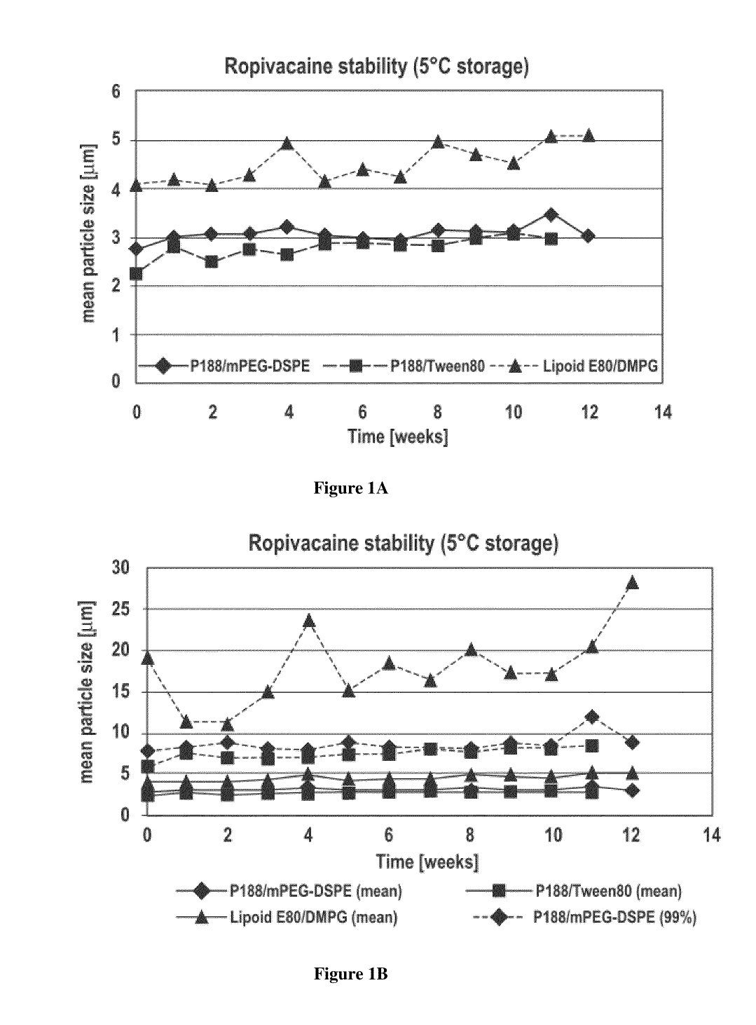

Formulations #1, #3 and #4 were also prepared at a slightly larger scale (40 mL), with all other conditions as described above, and stability testing was performed. The stability data for Formulations #1, #3, and #4 is shown in FIGS. 1A and 1B. The mean particle size of the microparticles in the dispersions was found to be stable when stored for 12 weeks at 5±3° C.

The solubility of Formulation #1 (i.e., the micro particles of Formulation #1) was assessed in both human plasma and phosphate buffered saline (PBS). Three tubes for each media were prepared by transferring 1 mL of either plasma or PBS into microcentrifuge tubes and adding 120 μL of the Formulation #1 suspension. The tubes were inverted to mix. All six tubes were visually turbid at the start of the experiment. The tubes were then placed on a spinner and incubated at 37° C. for 4 days. When the tubes were removed from spinner they were still visually turbid. The samples were centrifuged to separate remaining solids, and 100 μL of each supernatant was submitted for ropivacaine quantitation by HPLC. The solubility of Formulation #1 (average of three replicates) was 0.34 mg/mL in PBS and 0.48 mg/mL in plasma.

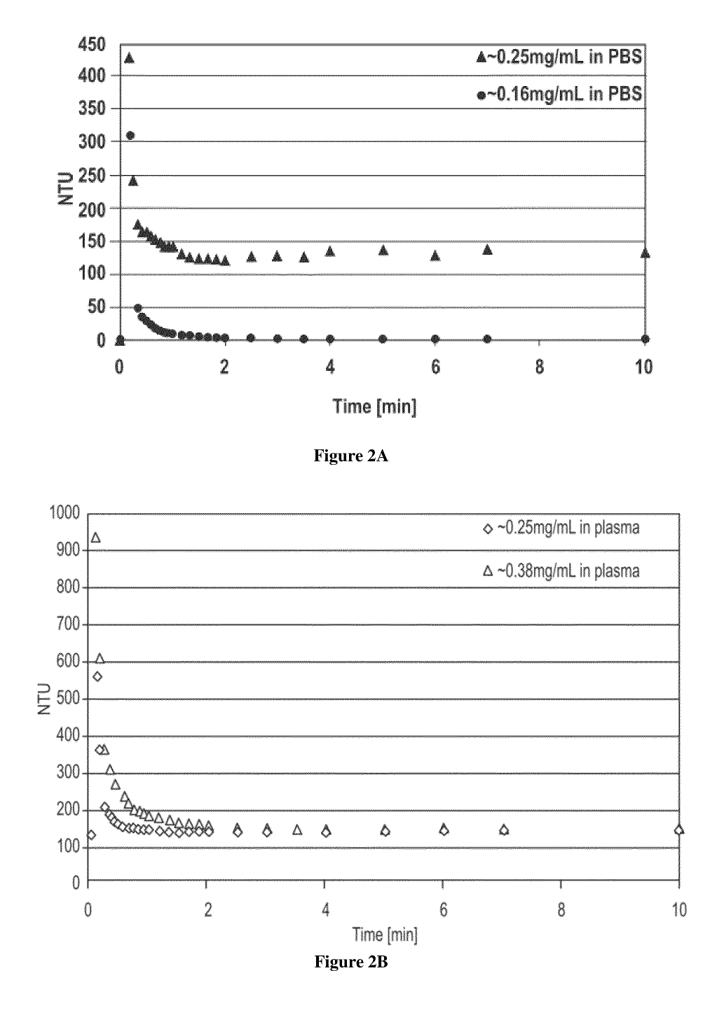

The dissolution profile of Formulation #1 was assessed using a turbidimetric method. Dissolution in both human plasma and PBS was evaluated. Briefly, 25 mL of either plasma or PBS were added to a turbidimeter vial and the baseline turbidity of each media was measured using a HACH 2100AN laboratory turbidimeter (Hach Co.). Then, varying amounts of Formulation #1 were added and dispersed in the media using an overhead stirrer for five seconds to create ropivacaine concentrations from about 0.16 mg/mL to about 0.38 mg/mL. Nephelometric turbidity unit (NTU) measurements were taken at various intervals until the readings either returned to baseline or did not change significantly. After the last measurement, each vial was removed and inspected visually. Vials containing ropivacaine microdispersions in plasma contained clear solution. For PBS, the vial at the higher ropivacaine concentration (about 0.25 mg/mL) was found to be visually turbid, whereas, the vial at the lower ropivacaine concentration (about 0.16 mg/mL) contained clear solution. The dissolution profiles of various concentrations of ropivacaine in PBS and plasma are shown in FIG. 2.

From FIG. 2A, the PBS solubility limit for ropivacaine microparticles apparently lies between 0.16 mg/mL and 0.25 mg/mL. The dissolution curve for the lower concentration plateaus at a nephelometry reading of 0, indicating complete dissolution to yield a clear solution. The higher concentration, however, plateaus at a turbid level of about 130NTU, indicating persistence of undissolved microparticles. The greater lipid binding capabilities of plasma for the hydrophobic drug are revealed in FIG. 2B. At both 0.25 and 0.38 mg/mL drug concentrations, the dissolution curves plateau to the original level of about 120 NTU, indicating no additional turbidity contributed by persisting undissolved microparticles. Plasma is normally more turbid than is PBS because of the presence of proteins.

### Example 2

### Microparticle Dispersion of an Analgesic Agent is a Sustained Release Formulation

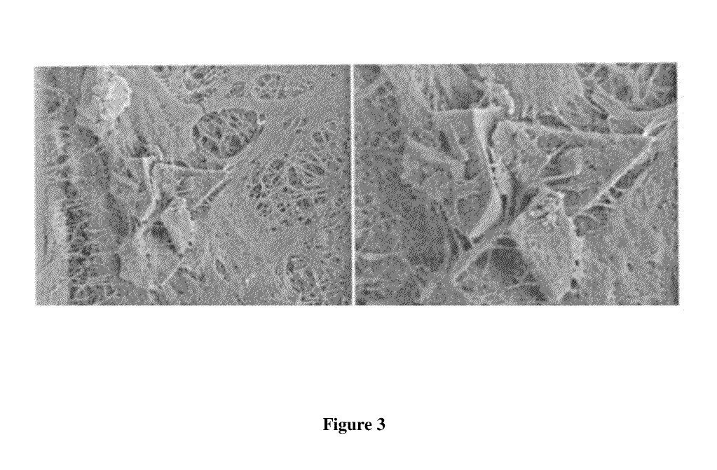

The release of ropivacaine from a sustained release fibrin matrix formulation (Tisseel®) was evaluated. Whole, human, citrated blood was centrifuged at 700 rcf for 15 minutes. The pellet was discarded, and the supernatant (plasma) was used for the release assay. Three aliquots of 250 μL of 10 mg/mL ropivacaine free base microparticles (2.5 mg) were prepared for Formulation #1 described above. The aliquots were centrifuged at 8,000 rpm for 5 minutes at 5° C. in 1.5 mL polypropylene microcentrifuge tubes. The supernatants were removed and discarded. The 2.5 mg of ropivacaine free base microparticles was combined with 25 μL of 100 mg/mL fibrinogen and 20 μL of thrombin dilution buffer. Next, 5 μL of 20 IU/mL of thrombin was rapidly mixed into the ropivacaine free base microparticles and fibrinogen. The matrices were allowed to polymerize for two hours before being transferred to 15 mL polypropylene conical tubes. FIG. 3 shows ropivacaine microparticles of Formulation #1 within the fibrin matrices. The matrices were then covered with 4 mL of human plasma. One 100 μL plasma sample was removed from each of the three tubes to serve as the baseline samples. The three tubes were incubated at 37° C. with orbital shaking for eight days. A 100 μL plasma sample was taken from each of the three tubes at one-, two- and three-hour initial time points. Subsequent samples were collected each day. The amount of ropivacaine in each sample was determined by mass spectrometry.

For ropivacaine, target parameters of efficacy can be determined from the published tissue drug levels of a slow release depot dosage form of bupivacaine found to alleviate pain at a minimum level of 30 μg/mL (McDonald et al., Pharm. Res. 2002; 19: 1745-52; Kopacz et al., Anesth. Analg. 2003; 97:124-31). The equipotent concentrations of bupivacaine and ropivacaine are 1 and 0.95-1, respectively (Practical Management of Pain, 3^{rd} Ed, P Prithvi Raj, ed. 2000, Mosby, Philadelphia, p. 561). Bupivacaine literature is therefore relevant for ropivacaine, and a target tissue drug level of 30 ug/mL of ropivacaine is sufficient to achieve efficacy.

The ropivacaine release rate (Ro) out of the Tisseel® formulation should equal the desired steady state tissue concentration (Css) times the drug clearance rate (CL) from the tissue. (M. Rowland and T. Towzer, Clinical Pharmacokinetics: Concepts and Applications, 2nd ed. Lea & Febiger, Philadelphia, 1989, p. 65). For example:

Ro(mg/d)=Css(mg/mL)×CL(mL/d)

The desired steady state tissue concentration is the desired interstitial steady state tissue concentration, i.e., 30 μg/mL for ropivacaine. The drug clearance rate is the drug clearance rate from the tissue and can be estimated from reported wound drainage fluid rates (see Table 2 below), because when used surgically, Tisseel® is placed in the tissue above the incision line.

TABLE 2

| | Wound Drainage | |
|---|---|---|
| Surgical Procedure | Rate or Volume | |
| Caesarian Section | 30 ± 20 | mL/16 h |
| Home discharge criterion | <20 | mL/24 h |
| Mastectomy | 600 | mL/6 d |
| Axillary lymph node dissection | 600 | mL/6 d |
| Thyroidectomy | 78 | mL/3 d |

Using the maximum wound drainage fluid rate of 4 mL/h and minimum wound drainage fluid rate of 1 mL/h from the table above, maximum and minimum drug release rates were determined:

Maximum:Ro=30 μg/mL×4 mL/h=120 μg/h

Minimum:Ro=30 μg/mL×1 mL/h=30 μg/h

The ropivacaine concentrations in plasma release from the Tisseel® matrices at 1, 2, 4, and 24 hours are in Table 3.

TABLE 3

| Time | Average Drug Concentration |
|---|---|
| 1 h | 118 μg/mL |
| 2 h | 154 μg/mL |
| 3 h | 173 μg/mL |
| 24 h | 300 μg/mL |

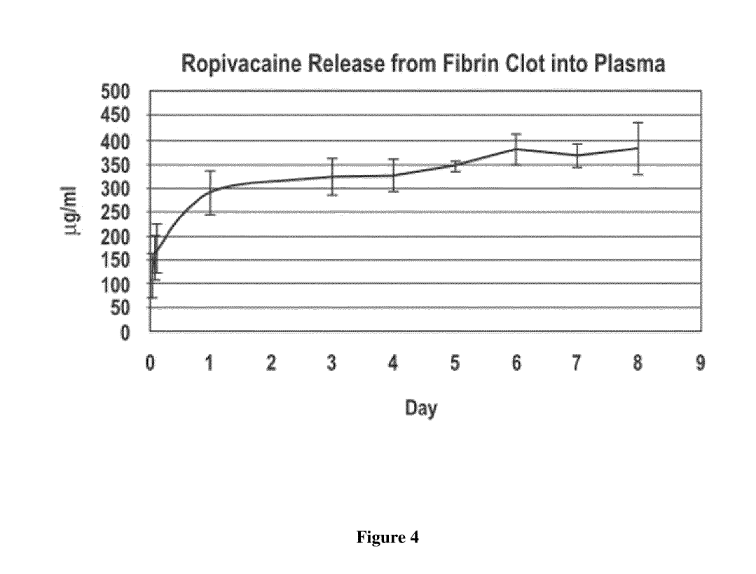

Over the next 6 days, the ropivacaine concentration in the plasma surrounding the matrix remained at 300 to 340 μg/mL, as shown in FIG. 4. Over the eight days, 1.1 mg±0.16 mg of the 2.5 mg of ropivacaine microparticles was recovered from the plasma. For the drug release rate, there was a burst release initially within the first hour providing 118 μg/mL, and then release at roughly the rate of 30 μg/mL×4 mL/h or 120 μg/h thereafter. The experiment demonstrated an adequate release rate to accommodate the maximum wound drainage fluid rate calculated above. At wound drainage rates less than this, the drug concentration will be constrained to an absolute maximum of 300 μg/mL, which is the static equilibrium (no flow) limit. The apparent solubility limit of ropivacaine released from the fibrin matrices was approximately 340 μg/mL, similar to the in vitro solubility described in Example 1 for the microparticles themselves, demonstrating that the drug was effectively released from the sustained release formulation.

The amount of drug available for a 3-day sustained release formulation was calculated as follows:

L(mg)=Ro(mg/d)×Duration(d)

where L is the load of drug (mg) to be determined

Ro is drug release rate, as set out above to be 120 μg/h×1 mg/1000 μg×24 h/d=2.88 mg/d
Therefore, L=2.88 mg/d×3 d=8.64 mg.

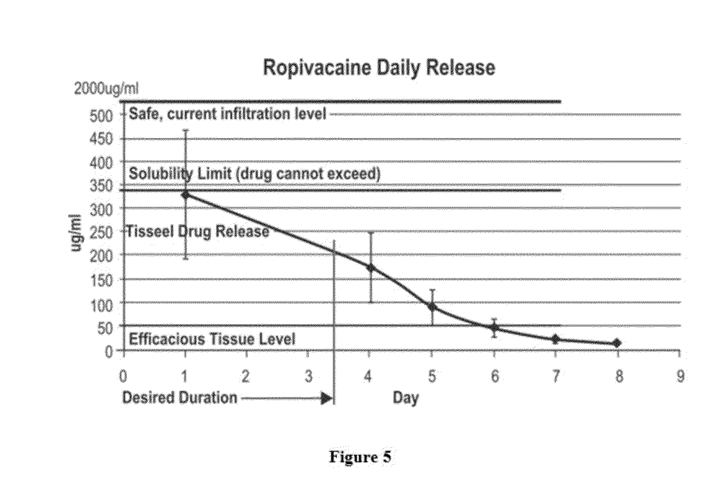

FIG. 5 shows the expected release of ropivacaine from the sustained release formulation. Ropivacaine is currently used in wound filtration at a concentration of 2000 μg/mL and such a level is considered safe. The amount of drug released over three days was calculated to be less than currently administered concentrations of ropivacaine and greater than the target tissue level required for efficacy, demonstrating that a microparticle dispersion of ropivacaine in a sustained release formulation according to the invention would be safe and effective.

### Example 3

### Administration of an Analgesic Agent Microdispersion to Reduce Pain and Inflammation

The ability of a dispersion comprising microparticles of an analgesic agent according to the invention to mediate pain and inflammation was investigated in an animal model. Lewis rats were inoculated with peptidoglycan-polysaccharide (PG-PS) (5.4 mg/mL, Lee Laboratories) by intraarticular injection in the right knee two weeks prior to the start of the examination period, i.e., on Day −14. PG-PS induces inflammation highly representative of that produced by certain drugs and is also used in models of arthritis. On Day 0, animals were dosed via single intraarticular injection of 70 μL saline, daily oral celecoxib 10 mg/kg, or single intraarticular injection of 70 μL (8.5 mg) ropivacaine suspension. Two hours post-dosing, the inflammatory response was activated by a tail vein injection of 0.5 mL of 0.4 mg/mL PG-PS. A control group did not receive the activation injection. The animals in the saline and ropivacaine groups were re-dosed on Day 23, after the initial inflammatory response subsided, and the inflammatory response was reactivated on Day 28 by tail vein injection. The oral celecoxib was dosed 10 mg/kg daily throughout the study except for Day 29, when the dose was increased to 50 mg/kg. The test groups are described in Table 4:

TABLE 4

| | Test/Control | Route of | | Dose |
|---|---|---|---|---|
| Group | Article | Administration | Dose | Frequency |
| 1 | PG-PS priming, no | N/A | N/A | N/A |
| | reactivation | | | |
| | (control) | | | |
| 2 | Saline (control) | Intraarticular | 70 μL | Day 0 and Day |
| | | | | 23 |
| 3 | Celecoxib | Intraarticular | 70 μL, 10% | Day 0 and Day |
| | suspension | | suspension | 23 |
| 4 | Celecoxib | Intraarticular | 70 μL, 5% | Day 0 and Day |
| | suspension | | suspension | 23 |
| 5 | Celecoxib solution | Oral | 10 mg/kg/day, | Daily |
| | (control) | | increased to 50 mg/kg/ | |
| | | | day on Day 29 | |
| 6 | Ropivacaine | Intraarticular | 70 μL, 10% | Day 0 and Day |
| | suspension | | suspension | 23 |

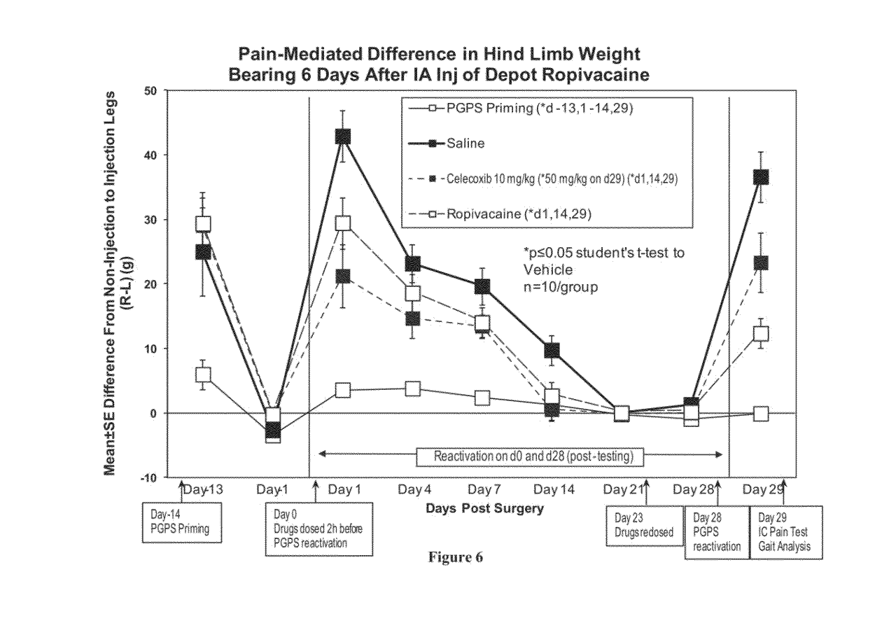

Pain was assessed using incapacitance testing and gait analysis at several timepoints throughout the study. During the incapacitance testing, the amount of weight borne on the experimental limb that received the PG-PS intraarticular injection was compared to the control limb as a measure of the animal's discomfort. FIG. 6 shows the pain-mediated difference in hind limb weight bearing. The rats that received ropivacaine (Group 6) showed significantly less pain on Days 14 and 29 (p≦0.05) than animals that received saline injections.

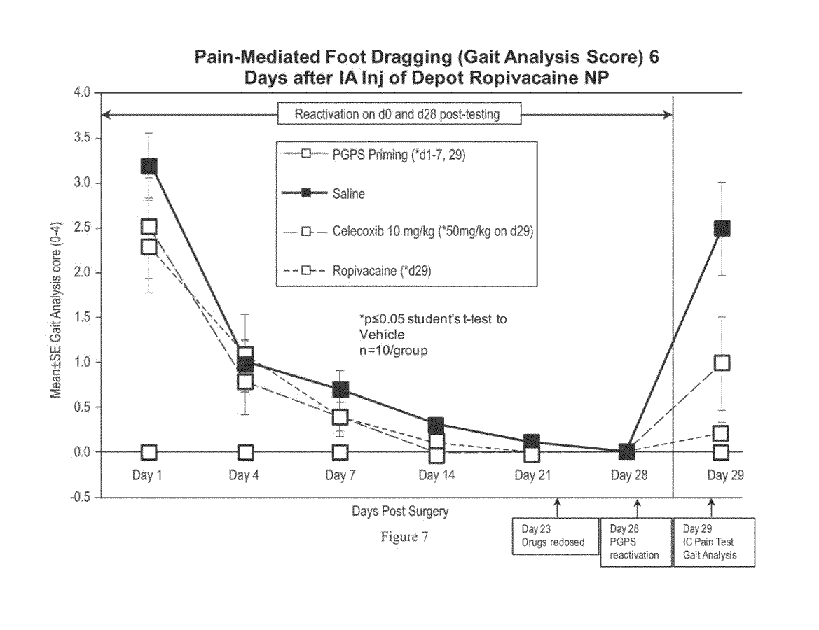

Gait analysis investigating whether the tendency of the animals to drag the injected foot corroborated that the ropivacaine had an analgesic effect (FIG. 7). Ropivacaine treatment statistically significantly reduced foot dragging when injected 6 days prior to test, indicating that the ropivacaine treatment nearly eliminated the pain completely. From the results of the hind limb weight bearing and gait analysis measurement, celecoxib clearly was more effective than the saline control. However, ropivacaine was surprisingly more effective than celecoxib.

Drug quantitation was performed on blood and plasma samples to assess the pharmacokinetic profiles of the drugs and the levels to which the drugs were present systemically. Knee joint tissues were also assayed for drug concentration in order to determine if any residual drug was present in the joints at the conclusion of the study. In addition, histological analysis and cytokine analysis were performed on knee joint tissues collected during necropsy on Day 30. Bioanalytical analysis of rat blood samples showed systemic levels of celecoxib that persisted for approximately 2 weeks after intraarticular injection. Quantitation of celecoxib was performed on plasma and whole blood samples collected from the rats in Groups 2 to 5 at timepoints of 1, 6, 12, and 24 hours, and at 7, 14, 21, and 29 days. In the Group 3 rats, measurable levels of celecoxib were detected in the blood up to and including the 14 day interval. The Group 4 rats also showed measurable levels up to and including the 14 day interval and on Day 29; however, the levels were systematically lower for Group 4 as compared to Group 3. Celecoxib levels in Group 2 (saline control) were primarily below the quantitation limit, as expected. Celecoxib was also detected in the knee tissues from Groups 3, 4, and 5, with the Group 3 levels being the highest, followed by Group 4, and finally Group 5. In contrast, bioanalytical analysis of whole blood from rats in Group 6 indicated that systemic levels of ropivacaine were only detected up to and including 24 hours. Ropivacaine was undetected in the rat knee tissues. Thus, the pain management effect of ropivacaine surprisingly and unexpectedly persisted at least to Day 7 despite the fact that the drug was undetected after the end of Day 1.

Histopathological examination revealed that the stifle joints in the rats receiving celecoxib (Groups 3 to 5) were similar to the saline control group (Group 2) and showed diffuse, moderately severe, subacute inflammation of the synovium. In contrast, intraarticular treatment with ropivacaine suspension was surprisingly and unexpectedly associated with a substantially reduced inflammatory response in joints from half of the rats in Group 6. The observed synovial inflammation on a scale of 1 (no inflammation) to 3 are summarized in Table 5.

TABLE 5

| Group | Animal 1 | Animal 2 | Animal 3 | Animal 4 |
|---|---|---|---|---|
| No reactivation | 1 | 1 | 0 | 0 |
| Saline | 3 | 3 | 3 | 3 |
| 10% Celecoxib | 3 | 3 | 3 | 3 |
| 5% Celecoxib | 3 | 3 | 3 | 3 |
| Oral Celecoxib | 3 | 3 | 3 | 2 |
| Ropivacaine | 1 | 2 | 3 | 1 |

To gain additional insight into the mechanism by which intraarticular ropivacaine microparticles minimized pain and tissue inflammation, tissue cytokine analyses were performed. There was a trend for the ropivacaine group, alone among all treatment groups, to have values similar to the control (Group 1) for IL-18. This suggested that the level of inflammation of a ropivacaine microparticle-treated animal was reduced to a normal state. There was also a trend for ropivacaine to yield significantly less IL-1b than the celecoxib-containing treatments. A reason for the apparently significant benefits of ropivacaine may lie in its inhibition of release of Substance P, which is known to increase IL-18, IL-6, and IL-1b cytokine expression. There was a trend for the ropivacaine-treated animals to be similar to the control (Group 1) for Substance P, suggesting a return to near normal levels for ropivacaine microparticle-treated animals. Thus, the study showed that the administration of a dispersion comprising ropivacaine microparticles conferred sustained relief of pain and inflammation in vivo. Unexpectedly, there was a statistically significant difference for the IL-18 and Substance P levels between the ropivacaine and celecoxib groups, further confirming the surprisingly effective anti-inflammatory activity of the analgesic agent ropivacaine.

### Example 4

### Co-Administration of a Primary Therapeutic Agent Microparticle Dispersion and an Analgesic Agent Microparticle Dispersion

The safety and efficacy of anti-HIV drugs co-administered with a microparticle dispersion of ropivacaine are evaluated. Dispersions comprising microparticles of antiretroviral drugs are prepared, for example, according to Dash et al., AIDS. 2012, 26: 2135-2144, Roy et al., J Infectious Diseases. 2012; 206:1577-88, and/or Balkundi et al., Int J Nanomed. 2011; 6:3393-3404. Briefly, dispersions comprising free base antiretroviral drugs such as atazanavir, ritonavir, and/or efavirenz, poloxamer-188 (P188) and optionally 1,2-distearoyl-phosphatidyl-ethanolamine-methyl-polyethyleneglycol conjugate-2000 (mPEG2000-DSPE) are prepared by high-pressure homogenization or wet milling. Particles are lyophilized and resuspended in saline prior to injection. Dispersions comprising microparticles of free base ropivacaine are prepared as described above.

Dispersions comprising microparticles of an antiretroviral agent and microparticles of free base ropivacaine are administered via subcutaneous injection or implantation into lab animals infected with HIV, for example, mice, rats, rabbits, and/or monkeys. Control groups receive injections or implantations of the antiretroviral agent alone. The presence of injection site inflammation and/or immunologic reaction is evaluated by incising the skin and macroscopically examining the injection site for alterations of normal structure, including necrosis, discolorations, infections, and/or encapsulation. To assess inflammation, five morphologic features are evaluated: endothelial loss, thrombosis, perivascular inflammation, perivascular edema, and perivascular hemorrhage. Endothelial loss is graded based on estimates of the relative thickness of the endothelium. Thrombosis is graded based on the relative size of the thrombus and degree of vascular lumen obstruction. Inflammation and hemorrhage around the site of administration are graded based on the number and distribution of leukocytes and erythrocytes, respectively. Encapsulation, if present, is measured by recording the width of the capsule from the periphery of the space occupied. The injection or implantation of the dispersion comprising antitretroviral microparticles alone results in injection site inflammation and/or immunologic reactions, however, co-administration of ropivacaine microparticles with antiretroviral particles reduces the adverse effects.

Blood and tissue samples are analyzed to assess viral activity. Despite reducing the inflammation and/or immunologic reactions associated with the administration of the antitretroviral microparticle dispersions, the co-administration of microparticles of ropivacaine does not adversely affect the efficacy of the antiretroviral drug, as confirmed through evaluation of efficacy endpoints including HIV viral load, antigen markers in the blood, drug levels in blood and tissue, immune status, and/or CD4+/CD8+ ratio. The experiment shows that a dispersion comprising microparticles of an analgesic agent can be administered to reduce the pain, inflammation, or immunological reactions associated with parenteral administration of a primary therapeutic agent, without influencing the efficacy of the primary therapeutic agent, thereby improving the therapeutic utility of the primary agent.

### Example 5

### Co-Administration of a Primary Therapeutic Agent and an Analgesic Agent Microparticle Dispersion

The safety and efficacy of a drug associated with injection site pain and/or an immunological reaction co-administered with a microparticle dispersion of ropivacaine is evaluated. An approved protein or peptide primary therapeutic agent that is known to cause an adverse antigenic response in a significant population, such as anakinra (Kineret®, Swedish Orphan Biovitrum), is injected subcutaneously into a non-human or human subject. Dispersions comprising microparticles of ropivacaine are prepared as described above, and the ropivacaine microparticles or a saline control is co-administered subcutaneously with the primary therapeutic agent. Subjects receiving the co-administered protein or peptide primary therapeutic agent and saline experience pain at the injection site and/or an adverse antigenic response. The adverse antigenic response is assessed by evaluating the subject for symptoms of an allergic reaction, including swelling, difficulty breathing or swallowing, or hives. Subjects receiving a co-administration of ropivacaine microparticles report a reduction in injection site pain and demonstrate a significant reduction in the severity and duration of the adverse antigenic response compared to the control group.

### Example 6

### Identification of an Anti-Inflammatory Analgesic Agent

A selected primary therapeutic is administered via subcutaneous injection or implantation into lab animals, for example, mice, rats, rabbits, and/or monkeys, or human subjects. Following administration of the primary therapeutic, an inflammatory response is triggered in a significant number of subjects because of an injection site reaction. Depending upon experimental conditions of the animal model, the selected primary therapeutic, etc., the time at which a strong, preferably maximal, inflammatory response is noted is used to define the measurement point in the experimental protocol. A study is designed, wherein Group A receives neither primary therapeutic agent nor any treatment; Group B receives the primary therapeutic agent (parenterally) and only saline treatment (parenterally); Group C receives the primary therapeutic agent (parenterally) and a dispersion comprising an analgesic agent candidate (parenterally); and optionally Group D receives the primary therapeutic agent (parenterally) plus a dispersion comprising ropivacaine particles (parenterally). Tissues samples are obtained at the predetermined timepoint corresponding to robust formation of inflammation of Group B. The levels of Substance P and/or IL-18 in the samples are measured using enzyme-linked immunosorbent assay (ELISA) or other analytical methods. Alternatively or in addition, levels of cytokines that are downstream of Substance P and IL-18 in the inflammatory cascade, such as IL-6 and IL-1b, are measured. Preferred analgesic agents minimize the precipitous rise in cytokine levels, resulting in attenuated increases of Substance P, IL-18, and/or other target cytokines to within 100% of normal Group A levels. The experiment is controlled for no inflammation, Group A; untreated inflammation, Group B; and can optionally be further standardized relative to ropivacaine-treated inflammation, Group D.

The foregoing Examples are provided to further illustrate the invention without being limiting. While particular embodiments of the present invention have been illustrated and described, it would be obvious to those skilled in the art that various other changes and modifications, can be made without departing from the spirit and scope of the invention. It is therefore intended to cover in the claims all such changes and modifications that are within the scope of this invention.

1. A method of reducing the pain, inflammation, and/or immunological reaction associated with parenterally administering a primary therapeutic agent, the method comprising parenterally administering to a subject in need thereof a therapeutically effective amount of a dispersion comprising microparticles of the primary therapeutic agent, the dispersion further comprising microparticles of an analgesic agent in an amount effective to reduce the pain, inflammation, and/or immunological reaction associated with parenterally administering the primary therapeutic agent, wherein the microparticles of the primary therapeutic agent and the microparticles of the analgesic agent have an effective particle size of less than 20 micrometers.

2. A method of reducing the pain, inflammation, and/or immunological reaction associated with parenterally administering a primary therapeutic agent, the method comprising parenterally co-administering to a subject in need thereof a therapeutically effective amount of a first dispersion comprising microparticles of the primary therapeutic agent and a second dispersion comprising microparticles of an analgesic agent, wherein the second dispersion is administered in an amount effective to reduce the pain, inflammation, and/or immunological reaction associated with parenterally administering the primary therapeutic agent, wherein the microparticles of the primary therapeutic agent and the microparticles of the analgesic agent have an effective particle size of less than 20 micrometers.

3. A method of reducing the pain, inflammation, and/or immunological reaction associated with parenterally administering a primary therapeutic agent, the method comprising parenterally co-administering to a subject in need thereof a therapeutically effective amount of the primary therapeutic agent and a dispersion comprising microparticles of an analgesic agent, wherein the dispersion comprising microparticles of an analgesic agent is administered in an amount effective to reduce the pain, inflammation, and/or immunological reaction associated with parenterally administering the primary therapeutic agent, wherein the microparticles of the analgesic agent have an effective particle size of less than 20 micrometers.

4. (canceled)

5. (canceled)

6. (canceled)

7. The method of claim 3, wherein the primary therapeutic agent is administered in a form selected from the group consisting of solutions, emulsions, liposomes, microparticle dispersions, implants, and combinations thereof.

8. The method of claim 3, wherein the microparticles of the primary therapeutic agent and/or the microparticles of the analgesic agent have an effective particle size of less than 1 micron.

9. The method of claim 3, wherein the primary therapeutic agent and/or the analgesic agent are administered in the form of a depot injection.

10. The method of claim 3, wherein the analgesic agent is selected from the group consisting of antihistamines, mast cell stabilizers, corticosteroids, anti-inflammatories, local anesthetics, and combinations thereof.

11. The method of claim 3, wherein the analgesic agent is selected from the group consisting of lidocaine, mepivacaine, prilocalne, etidocaine, bupivacaine, levobupivacaine, ropivacaine, dibucaine, articaine, cocaine, procaine, mepivacaine, prilocalne, articaine, benzocaine, chloroprocaine, etidocaine, tetracaine, dibucaine, butamben, capsaicin, their salts, hydrates, prodrugs, and combinations thereof.

12. The method of claim 3, wherein the analgesic agent is ropivacaine.

13. (canceled)

14. The method of claim 3, wherein the primary therapeutic agent is a drug selected from the group consisting of peptides, proteins, antibodies, anti-retroviral drugs, and combinations thereof.

15. The method of claim 3, wherein the primary therapeutic agent comprises an anti-retroviral drug.

16. (canceled)

17. (canceled)

18. (canceled)

19. (canceled)

20. (canceled)

21. (canceled)

22. The method of claim 3, wherein the co-administration is via intrarticular injection, intradermal injection, subcutaneous injection, and/or intramuscular injection.

23. The method of claim 3, wherein the microparticles of the analgesic agent are incorporated in a matrix, the matrix optionally further comprising microparticles of the primary therapeutic.

24. The method of claim 3, wherein the dispersion comprising microparticles of the analgesic agent is a sustained release formulation.

25. (canceled)

26. The method of claim 3, wherein parenterally administering the primary therapeutic agent renders the subject susceptible to an adverse antigenic response.

27. (canceled)

28. (canceled)

29. A pharmaceutical composition comprising a dispersion comprising microparticles of an analgesic agent in an amount effective to reduce the pain, inflammation, and/or immunological reaction associated with parenterally administering a primary therapeutic agent, wherein the microparticles of the analgesic agent have an effective particle size of less than 20 micrometers.

30. (canceled)

31. A pharmaceutical composition comprising a fibrin matrix and microparticles of an analgesic agent, said microparticles being dispersed within the fibrin matrix, wherein the microparticles of the analgesic agent have an effective particle size of less than 20 micrometers.

32. (canceled)

33. (canceled)

34. The composition of claim 31, wherein the composition further comprises microparticles of a primary therapeutic agent, said microparticles of the primary therapeutic agent being dispersed within the fibrin matrix, wherein the microparticles of the primary therapeutic agent have an effective particle size of less than 20 micrometers.

35. A method of preventing or reducing pain, inflammation, and/or immunological reactions in a subject suffering from arthritis, the method comprising delivering a composition according to claim 31 proximate to a site of arthritis, said composition being capable of releasing the analgesic agent in an amount effective for preventing or reducing pain, inflammation, and/or immunological reactions at the site of arthritis.

36. A method of preventing or reducing pain, inflammation, and/or immunological reactions at a site of surgery or at a wound site in a subject in need thereof, the method comprising delivering a composition according to claim 31 proximate to the site of surgery or the wound site, said composition being capable of releasing the analgesic agent in an amount effective for preventing or reducing pain, inflammation, and/or immunological reactions at the site of surgery or the wound site.
